SEQUENCE LISTING (31 sequences)

SEQ ID NO: 1 - DNA, 2450 bp, Homo sapiens
  CDS (155)..(1924)
  Unsure (58)..(58): n=a,c,g,t any unknown or other
aattccttttttttttttttttttggaattccgagacagagtcgcggcgcagggggcngtccccggccgggacgcgggtcgcgtcgttgtcctccgcgagcgtccggattgcaggctgtctgtccccagaccccagagcacgtccagcaccaccatgactgggctgttgaagaggaaatttgaccagctggatgaggacaactcctcggtctcctcctcctcctcttcctctgggtgccagtctcgctcctgctccccaagctcttctgtctcccgtgcctgggactcagaggaggaaggcccctgggatcagatgcccctgcctgaccgtgacttctgcggccccagaagtttcacccccctgtctatcctgaagcgggctcgccgggagcgcccaggccgtgtagcctttgatgggatcaccgtcttctacttcccccgctgccagggcttcaccagtgtgcccagccgtggtggctgtactctgggtatggcccttcgccacagtgcttgccgtcgcttctctttggctgagtttgcgcaggagcaagcccgtgcacggcacgagaagctccgccagcgcttgaaagaggagaagttggagatgctgcagtggaagctttcggcagctggggtaccccaggcagaggcagggctgccacctgtggtggatgccattgatgacgcctctgtggaggaggacttggcagtcgctgtggcaggtggccggttggaagaagtgagcttcctacagccctacccagcccggcgacgtcgagctctgctgagggcttcaggtgtgcgaaggatcgatcgggaggagaagcgggagctgcaggcactgcgccaatcccgggaggattgtggctgtcactgcgataggatctgcgaccctgagacctgcagctgcagcctggcaggcatcaagtgccagatggaccacacagcattcccctgtggctgctgcagggagggctgtgagaaccccatgggccgtgtggaatttaatcaggcaagagttcagacccatttcatccacacactcacccgcctgcagttggaacaggaggctgagagctttagggagctggaggcccctgcccagggcagcccacccagccctggtgaggaggccctggtccctactttcccactggccaagccccccatgaacaatgagctgggagacaacagctgcagcagcgacatgactgattcttccacagcatcttcatcagcatcgggcactagtgaggctcctgactgccccacccacccaggcctgcctggccctggcttccagcctggcgttgatgatgacagcctggcacgcatcttgagtttcagtgactctgacttcggtggggaggaggaggaagaggaggaagggagcgtggggaacctggacaacctcagctgcttccatccagctgacatctttggtactagtgaccctggtggcctggccagctggacccacagctattctggctgtagcttcacatcaggcgtcctggatgagaatgccaacctggatgccagctgcttcctaaatggtggccttgaagggtcaagggaaggcagccttcctggcacctcagtgccacccagcatggacgctggccggagtagctcagtggatctcagcttgtcttcttgtgactcctttgagttactccaggctctgccagattatagtctggggcctcactacacatcacagaaggtgtctgacagcctggacaacatcgaggcacctcacttccccctgcctggcctgtctccacctggggatgccagcagttgcttcctggagtccctcatgggcttctccgagccagccgccgaagccctagatccctttattgacagccagtttgaggacactgtcccagcatctctaatggagcctgtgccggtgtgaggaccaggatgtcttttcccagccccaagagacctgttgctgctttcttgtaattatggggctccccagagtctgcgtaacagtctcccactggctggctcacccacaggtgccatgtgcacactcctggttttcaaacaattctctggatttatttatttgttttaacttttctgtgctgaagagaggactagggggagggggcttcccctttcagctgcccggccccccacacccacagcttgctcttctatctccacaacgtgagcctggaagaggagaaaatgtggctcctctggagcttggcagaccacttttcggtctttgcgtgatgttccttagcccaaagacggtgagacagggctgaaatcaggtggcttctgccaccctgagccctagacccatgggtggctaaatccactggactgtgaagactataatttatttccataatttatttggagattgaggaggctttggttgcacttctttggctggtgggtaatgccaggggtggggtgggcnc

SEQ ID NO: 2 - protein, 589 aa, Homo sapiens
MTGLLKRKFDQLDEDNSSVSSSSSSSGCQSRSCSPSSSVSRAWDSEEEGPWDQMPLPDRDFCGPRSFTPLSILKRARRERPGRVAFDGITVFYFPRCQGFTSVPSRGGCTLGMALRHSACRRFSLAEFAQEQARARHEKLRQRLKEEKLEMLQWKLSAAGVPQAEAGLPPVVDAIDDASVEEDLAVAVAGGRLEEVSFLQPYPARRRRALLRASGVRRIDREEKRELQALRQSREDCGCHCDRICDPETCSCSLAGIKCQMDHTAFPCGCCREGCENPMGRVEFNQARVQTHFIHTLTRLQLEQEAESFRELEAPAQGSPPSPGEEALVPTFPLAKPPMNNELGDNSCSSDMTDSSTASSSASGTSEAPDCPTHPGLPGPGFQPGVDDDSLARILSFSDSDFGGEEEEEEEGSVGNLDNLSCFHPADIFGTSDPGGLASWTHSYSGCSFTSGVLDENANLDASCFLNGGLEGSREGSLPGTSVPPSMDAGRSSSVDLSLSSCDSFELLQALPDYSLGPHYTSQKVSDSLDNIEAPHFPLPGLSPPGDASSCFLESLMGFSEPAAEALDPFIDSQFEDTVPASLMEPVPV

SEQ ID NO: 3 - DNA, 1749 bp, Mus musculus
  CDS (1)..(1749)
  variation (187)..(189): r=purine  ( CAR encodes CAA (aminoacid Gln) or CAG (aminoacid Gln))
atgactgggctactgaagaggaagtttgaccagctggaagaggacgactccagttcctcctcatcgttctctagccggctctctctctcctccttccccgcctcttctgcctcccctgcctggaactctgatgaggagggaccaggtggtcaggcaccccagtctgatcaggactcctgtggcctccaragtttcactcccccgtccatcctgaagcgggctcctcgggagcgtccaggtcacgtggcctttaacggcatcaccgtctactatttcccacggtgccagggattcaccagtgtgcccagccgtggtggctgtaccctgggcatggcttctcggcacagcacctgccgccttttctccttagctgagtttacacaggagcaggtccgggctcggcgtgagaagctccgccgccgtttaaaggaggagaagctggagatgctgaggtggaagttttcagtggctggagttccggagtcaggggcaggcgtgcctctcacagcggatgccatcgatgacgcctctgtagaggaggacttggcagtggccgtggcaaatggccgcctggaggaagccaatttcctccagccccatccacctcggcagcgacgggctctccttcgggcttccggcgttcgaaggattgaccgagaggagaagcgggagctgcaggtgctgcgccagtcccgggaggattgtggttgtcactgtgatggcgtctgtgaccctgagacctgcagttgcagcttggccggcattaagtgccaggtgatggatcacacgtccttcccctgtggctgctgcagggagggctgcgagaaccccaatggtcgagtggaattcaatcagacaagagttcagacgcacttcattcacaccctcacccgcctgcagatggagcagggtgccgagagcttgggggacctggagtcccccgtggaggacactcctgttgaacaagccgcgctttccccctttccgccttccaagccccctgtgagcagtgaactgggggacagcagttgcagcagcgatatgacagactcttccacgaccttgtcctctggcagcagcgagcctccaaaccaccctgcccaccccagcctgccaggtcccagcttccggtctggcgtagatgaagacagcctggaacagatcctgaatttcagtgactctgacctcggaattgaggaggaggaggaggagggagggggtgtgggcaacttggataacctcagctgcttccatctggctgacatctttggtaccggtgaccccggcagcctggccagctggactcacagccagtctggctctagcctcgcatcgggcatcctagatgagaatgccaacctggacgccagttgcttcctgaacagcggacttggagggctgagagaaggtagcctccctggcagttctgggtcccctgagggggacgccgtccagagcagctcctgggacctcagcttatcctcctgtgactcctttgagcttctccaggctctgccagattatagtctgggacctcactatacttcccgaagggtgtcgggcagcccggatagccttgagaccttccacccattgcccagcttctctccaccacgggatgcgagcacttgcttcctggagtctctcgtgggcctgtctgagccggttacagaagtcctggcaccccttctggagagccagtttgaggatgctgccttggcacctttgttggagcctgtgccagtgtaa

SEQ ID NO: 4 - protein, 582 aa, Mus musculus
  Unsure (63)..(63): Xaa = Gln
MTGLLKRKFDQLEEDDSSSSSSFSSRLSLSSFPASSASPAWNSDEEGPGGQAPQSDQDSCGLXSFTPPSILKRAPRERPGHVAFNGITVYYFPRCQGFTSVPSRGGCTLGMASRHSTCRLFSLAEFTQEQVRARREKLRRRLKEEKLEMLRWKFSVAGVPESGAGVPLTADAIDDASVEEDLAVAVANGRLEEANFLQPHPPRQRRALLRASGVRRIDREEKRELQVLRQSREDCGCHCDGVCDPETCSCSLAGIKCQVMDHTSFPCGCCREGCENPNGRVEFNQTRVQTHFIHTLTRLQMEQGAESLGDLESPVEDTPVEQAALSPFPPSKPPVSSELGDSSCSSDMTDSSTTLSSGSSEPPNHPAHPSLPGPSFRSGVDEDSLEQILNFSDSDLGIEEEEEEGGGVGNLDNLSCFHLADIFGTGDPGSLASWTHSQSGSSLASGILDENANLDASCFLNSGLGGLREGSLPGSSGSPEGDAVQSSSWDLSLSSCDSFELLQALPDYSLGPHYTSRRVSGSPDSLETFHPLPSFSPPRDASTCFLESLVGLSEPVTEVLAPLLESQFEDAALAPLLEPVPV

SEQ ID NO: 5 - DNA, 13558 bp, Mus musculus
  Unsure (4501)..(4501): n=a,c,g,t any unknown or other
ggatcccaggtggcactcctccctcccttcttccctcccttcctccctctctctcctcagacccagccagggctccagcactcacgcctcaaacagcctggcctttccagaactctgcctgaagctttgaatgttaacgctcttcagccttagttcttatcttgggtacactcagctgtggatctctatccagtccacacctacctctctgacatctgtttactcttagggacccggagcctctgagatgccacctcacacttgcactcgagtgagcctgtccctgtctccttccccttccccccctaccccgtcctcaaagccacctttgaacactgttcttggctcagccagctctgaccctggctgggggatgggcatgtgtctcctcattagattagctttcagggccaggaaaactaagacccctgcctgggctaaagcactgcctgtgtcacagtcctgctgcccactcttctgggcctctctctgccaggcttattatggctatgcaaaaatagcctatagccaagccttgtgtgaggagaggcccgttacactgtggtcacttatagctcttaaagagttgaacccagggtttcaatgtgtgccagacagctgacctatgtccctggcacatatggaggcaaatgtctgccatcccagcacgtgggaagtgaacgaacactggaggatcaggatagaggccatcaagggctactcagactctctcaatcaagatctattatttcctaaagcacttgcctccaaggtcatgacatcggagattaatctttgatttgggacttttttttttttttttttttttttttttagaaagkgttctgttcatagcagtatccccaaaaggcagaagaagtgtgtgtrggagggtgggggggcttttgatgtccttttggaaataactttthkggagaagaaggtttttctctgtaaatcaggttggcttaaactccaggcaattctcctgcctcatttgagattggataaacatgagctatgatactcaactcagaaatgacttctggaagggtgtaagtcttggtgggctgttccagtctttgaagaaggtagggaggatagcagggacatggccttgtggggaactgcaaacagtggtgctcagcatgggaactgcaaacagtggtgctcaacatggggactgtaaacagtggcactcagcatggggactgtaaacagtggtgctcatgtgcaaacaatggagctcagcatggggactgcagacagtggtgctcaacatggggactgtaacagtggctgkcagttcagcatgstgttttctttctgcatttggcstgwggggkatagggaggmtrgaagagactagatggtagccaggtgggggcagacacagaacaggtgataagctccgtgtgtgtgctgagaagccacctcagggctttgcaggagcccttcagggagctccaccatcaggtgacctacatagaacaggaatacagtctaggaatgccactcctgcagtggaggaattgtgaagcatttaacwcccctgcaaacctacccaggccaacctctacccatcatcctacccccacccccacccccaggaaatcccctcacaactgtcccctggtcccacataccccctcctgggagattgtcctgtcctttcacctgactgtggagaaggcaggtacaatgccttctaattgggtctccccttttgtcaatctcccatttgcattttctgtcgagttcatgcctgtgtctccactcaggagtcctcgggcttactatcatttacacacttactgagcacacgattcactgactgaggacttgacatgtgtccattcctgtattccacccagccatgtgaggttctaatgtgtgtttatttccttcgagacgccgtctcccttagcggaggctgatcttgaactcttgattctcctgtacctccagagtactaggatagatatcaagtgtgcaccaccctggcttgccttctgggaccttcagacaattggagacagtgatgttgagggacctacccaggatcacccagccaataaccaaggcaaggtccaaatcaggcagttgattttggcctctaggatatgtcccaagtcggtgcctagcctgtaggtgaacttccaggactgagactgcctgggtgtgggtgcaagaagaggggagggagaagctgagggtggctgcaggctgcaggtttctggcttgagcaactgggtgggggctgcagttactaagacagggaacacagaagaggaacagatggcatgggaaggagcagagaggttcaatttggggcctgtgaaattgtgtatttccaggagaagtgggtccttgcttggtttgggggtcttggacttgtgcccttagtctcacaggaaacaactggaattagagagggaggtagttccccccctccctgtgcattaggctgtggtggtcgcaggatgtttagggggctacctgtcaacctgtgtgtatgaatgagtgtgtctgtgtaaggatgcgtgtgcccgtgtgtgtgatgcctggctcatggccaaacaccctcatctcctgggagagtctacagacaggttaactgggctgtttgtgtttgtggtttgactctgggaagccgggtaacctagcctgagaaactgccccggaaacacctgcgtgtccttcttcccccgggcctggggtgagggcttgacctggtagtaagattttcttgagcctctcccatgcctgcctggaggagatagatggctgttctccattgagtacttttatccagtgagttgacagctggcgccctccgatttagggacctgctgggaggtataggttaggagaagggaccacaggataccttggtgaaggagacccagacagctttctgcttagttttcctgcctctttcacttcctgtgatggtggtgggggaggagtccccagctgtggscattcaaccctgaatgagtgagtaaggcagmctcaagacagcgacmcagcaggtagctgcactcagccaccatccttgaaccactragggtctccaggtcctgraaggactgttacttgtytragcactggggggttaggaaagragctttaaccccctccagcctgactggtgagtgagtgcatcttacctaggtccagagaagctacaaaggagcaagggaaacgggagagcagcttttcctgatcagtttcactattagaggtttgttttgttttaaggagagttaaccataatggtgagctattgaaggtgaatttaagatcaaagtgaagtctcctgccacagtccagcctagagcaggggacttttctcctgtggcatgggggtttctgagggaaagtctaagctccatcccagggaggggaggggcttgggaaagtccttctgctgggaatccagccaacatcaaaaccagtgcccattctcagagctcctgttcagcctggcccaggagctgacctgaagctgtcagtgggcaaggcggtggacctgtgtggtgggagaggagcsccgctctgggggggggggggggtcgtctggagattgtagaaggccctgcacaggcaagtgcacaaagacccgagtgcctgtgagtcagcctccaggattcctgtgaagatgagccaggctagttctgtcagtccatctgttggtctgtctgcctgctggcctgtgtgtgtgtctggtgtatgaatgtgtgtaggtgtgtgcatgtatgaatgtgtaggtggcaatggggaaggtacctggctgttttccagagaaggcttgttatcaggggccaggtcttgggaggccaaagtgtctggaacttggatttgcgtgttaagactcatttgagggaggagatactactgtgttgattggggctggggaccctactggagacactaccatgaacctgctctggctgggctggagggctcagagtctgggagataccatggaaggtatgcaggggaaacatggctaggcagggagcctatctcttaatcgttccattatcttcatcccaggcgtctgtcctcggctgttggaaccccctacattccaccatgactgggctactgaagaggaagtttgaccagctggaagaggacgactccagttcctcctcatcgttctctagccggctctctctctcctccttccccgcctcttctgcctcccctgcctggaactctgatgaggagggaccaggtggtcaggcaccccagtctgatcaggactcctgtggcctccaragtttcactcgtgagtctcccatttcggmcccccccaaccctyccccttyttccttycctaaaactgaaattggbcgtttttggcctcggtagcccyctcarggtgcctccttytgagaagwcgtgtgactgctccactctgsagagacaccttgacttcctgttgaccgtgggggttgtgctgytggagtccgagactttgcttagagtnggctcgtggctctgsggccttasaggggwgttgcggatgtgtatgcscggcacamcctamcyattccaggaacaccactrgrggctcawccctctggcttccacccccccccctgcactaggcctgctaagcctcttccctctccctccattttagcttctttctgaatgtccccaaaagggtttttccagaatagttttcttctcttcctttttttgagagcacactctcatataatccaggttgccttccagctgtgtggcatctgcctcccaagtacagggattcgggatgcacgccaccatggctgacttcctggttatctttctctctttttttttctttctttttttaaagacttatttattttataatatagtgagtacacgctcactgtctttagacacaccakaagagggcatcagatccatttacagatggttgtgagccaccatgtggttgctgggaattgaattcaggacctctggaggagcagtcggtgctcttaacctctgagccatctcgccagccccctccctggttatctttcaagtttgtataattttgattttaaactgaaagatggtcttgatggyttttttttttttttttttttttttggcttttttcaagacaaggttctctgtatagccctggctgtcctggaactcactctgtagaccaggctggcctcgaactcagaaatccgcctgcctctgcctcccaagtgctgggattaaaggtgtgtgccactattgcccagcgatagtccattttttaaaaaaattatttctttttatttttgagattataattacatcatttcccctatcttcccctccctccaaatcttcccacatatccgtccttatcctctttcaaattcatggtctctttttttttttttaattattgttacataagtctatacagctatattcctacatatataactccagcctgctttgtttttataatgktaaaggcaacctttgattttcagcatgctctttctccttcaagaaccattttagacacttttcccctacaaggggaatgtttataaacacaggattacgtctccctatgaagttttgaaaccatagttgttccttccatgtctcctatacaacatacctgtgtgttgcatattttttaaaaaagtatgatttttaatgcctggaatactaacaggaaatttcttagaaggaattttctcaggtagttgagtacaaagttaatatgttaacttgaagtcacttggcaaacatataatcccagaaccggggaggcaggagggctggggttcaatgtcgcttgctgttgaaccagtcctagccagcttgggataaatgatggtgtatgcctttaattccagttcttgagagacaaagcctggtggatcaagaccaacctgggctacgtatcaagtcctcggacagtcagtgttacatagtgagaccttgtctcaagaaaacataaacaagggctagaaggatggcacagtagttaagagaatcttgctcttgcagaggacccaagtgtggtccttagcacccacatcaggatggctcaccaccacctgtagctcctagcactagggatctggcgctttctgaacttcaaaggcacccacacccaagtggcatacactcacacatgcacttattaaaaataagtaagtttgaattgggcagtggtggcgcatgcctttaatcccagcacttgggaggcagaggcaggtggatttctgagttcgaggccagcctggtctacagaatgagtttcaggacagccagggctacacagagaaactctgtctcagaaaaccgaaagagacagacagacacactttctgggtctgtttcagaagacttaaactataaaggaaaagaccaatgacacaaatatatatttttaatttatgtgcatgagtattttgcctgtggatatatgtctatgtaccatgagcttgttatgcctgaggaggccagaagagggcgttagagcttctgaaactgagtttcttggctgcgacctctcctgtgggtgccagggctcttaactgctgagccatttccccagttctttgatatatgtttaaacaggggattatattattacatgtaaagaataagaattacagaaaattgtacaaaggatttataaatccgtggtaagggtggcaagacgcttcatctttgcgggttcatttgtgctgagaaatgacagttaagctggtgttgggttgggggtggtattgttctccagatccagccaggcctggcttttaagttcagatccaaggtgggtggagaaagagaaggggttggaatctttctagagattataccacatcgagggaagctcaatcccttctcttttccctttcacccccgtccatcctgaagcgggctcctcgggagcgtccaggtcacgtggcctttaacggcatcaccgtctactatttcccacggtgccagggattcaccagtgtgcccagccgtggtggctgtaccctgggcatggcttctcggcacagcacctgccgccttttctccttagctgagtttacacaggagcaggtccgggctcggcgtgagaagctccgccgccgtttaaaggaggagaagctggagatgctgaggtggaaggtacgggaggtttgcacgttgcggagttgttggtgttaggggagattcaatggctgggctatagctcaggcagggagagcgcctcctagtggggaggaggacctggtgtccatcaccagcactgcataaaccaggtatcttggtgcacccatcgtcctggaacttgggtggtgaaagccgaggaatcagggattcgaggtcatcctccgcaagcttgaggccatggccctttcccaaatgaaagtagtcggcttcagaccacaggctaggccctgggcataaataatgcttgaatcttcctagctgaatccgatcagccggctagactgatctgacactgcccagtacatcggggtttgagtaagagatccttgtagctgtggagcacagtgtgtacccctcccccacagacagcaccccactctctgggagagctaaatctctctctctctcacaccgtctttctgtacttatagttttcagtggctggagttccggagtcaggggcaggcgtgcctctcacagcggatgccatcgatgacgcctctgtagaggaggacttggcagtggccgtggcaaatggccgcctggaggaagccaatttcctccagccccatccacctcggcagcgacgggctctccttcgggcttccggcgttcgaaggattgaccgagaggagaagcgggagctgcaggtgctgcgccagtcccgggaggattgtggttgtcactgtgatggcgtctgtgaccctgagacctgcagttgcagcttggccggcattaagtgccaggtgcgtggtcggactgggtaagataaggggggtgtgcctataatcccagtactcgggcagggctacacagggaagccctgtctgtggggagccgggcctgctggcttcttgcatctccgaagttctcactttggcttttctttctctgcagatggatcacacgtccttcccctgtggctgctgcagggagggctgcgagaaccccaatggtcgagtggaattcaatcagacaagagttcagacgcacttcattcacaccctcacccgcctgcagatggagcagggtgccgagagcttgggggacctggagtcccccgtggaggacactcctgttgaacaagccgcgctttccccctttccgccttccaagccccctgtgagcagtgaactgggggacagcagttgcagcagcgatatgacagactcttccacgaccttgtcctctggcagcagcgagcctccaaaccaccctgcccaccccagcctgccaggtcccagcttccggtctggcgtagatgaagacagcctggaacagatcctgaatttcagtgactctgacctcggaattgaggaggaggaggaggagggagggggtgtgggcaacttggataacctcagctgcttccatctggctgacatctttggtaccggtgaccccggcagcctggccagctggactcacagccagtctggctctagcctcgcatcgggcatcctagatgagaatgccaacctggacgccagttgcttcctgaacagcggacttggagggctgagagaaggtagcctccctggcagttctgggtcccctgagggggacgccgtccagagcagctcctgggacctcagcttatcctcctgtgactcctttgagcttctccaggctctgccagattatagtctgggacctcactatacttcccgaagggtgtcgggcagcccggatagccttgagaccttccacccattgcccagcttctctccaccacgggatgcgagcacttgcttcctggagtctctcgtgggcctgtctgagccggttacagaagtcctggcaccccttctggagagccagtttgaggatgctgccttggcacctttgttggagcctgtgccagtgtaaggattaaggtgccttcttcctgccctgagaccctgttgctgctctttgtgatcttggggttccccgaggtctatgtatgtaacggtctcctacgggctggtcctgctcatggtgccatgtgggcaccctctatttttacattaacactctgaatttatttatttttttatgtttttctgtactgaagaagggagggtggggagggtacccctctttcaatgcctggtctctttatgtccaaacagaggtcttccacctcctactgtatgcctgaaggaggaaggggcggggctctcctggagcctcgcagtccctctttctgtactgtaagatgctccttgctccaaagacagcgacggctgagcgcagggccttaggggcttctgtgaaccataggagctaggtcttctggactgtgaagatgtaatttatatctgtaatttatttggggatggagacggcagtggtggagcctctctggcgggtgggcaggcagtgttggggccgaggcttcggtgtaccctgccagtctgggcttcggtgtggtgtgtgggttcgagtttatggctgctctattaaacagcggcctggctcaactgagcagacagaggctgtgcttgaccagcggtgaccaaaaccccttctgcccccttcccagaaactctacttgccctctttccaattccctctccccaaggccatggattataaagcccctctgtaagatggtgagccaggggccctaagagggcatgaagcacaccctgatcactgtctcaggcctttctgggcactgactcgaccctggcccacctcacgcccccaggccagttggcaactggtggctcttgagggctcttacgcccttcattagctgatgttggattttatatgcatttttttattgtctctaagtgtcagaactataatttattcatttctttgtgtgtgtgtgtgtgccaagaaacgcaggctctgggcctgcctccttgcccaggaggccttgccagcctgtgtgcttgtgggaacacactgtacctgagctgacaggtaccaataaagatgctctatttttaagtgtggtcttcttggtgaggtggggagtgtggggtacccccaggaagcatgtggggtggggcactatcatgtagcatggacagaagctgtactgaggcaggaggacagcatgttgggctttcttccaggcgggccagtcaggctgtaggcagagagccagctgaggttggctgggcaagtggaacagttttttttttttcccctctgaattaacccattcctccatgatgccatctggagttgtaaggaggcagagctgtctgctcaggcccagggagggcagcctagcctagtggaggcctggcctggtgacaggctgctcaggcagctgtagtccctggggacttgagctccagaactgtctctacctgccagacctgccagacctgtcctgaggcagccagaatagggtttaaggtcaggggtgcagagcaggctgccgtaccccgttttggggaacctctgtctggagcctgactgctagtgatttgcacaggccctctggagaatgaccacaggagagccttcctgtgctaaagcccctctactgtattctacccagaagaatggatgcagtcccagctctcgctgcctagtgaggcataggttatgtagattgcctgcctagagcctggatctctccaatcccatgcaagcgattgctgctcctctcaaactggcttggttcatgtrccttgagtgcctgggtcatgattctgtttttatgtttgtgacctgcttttgacctgctttttctggatgtgacctcatggcaacaccagtgaccaccagggggccgaggacttggcgggctgcctgtagttgggcatgagtacctgtgactggataaagtgccctgcaacatcctgttgtcctgtcccctctcactggggctgggaaacgcttgggcagagatcgagctgggggtgcagcatcctgtgtctctcttccaaaggttctgagaggtttctctggtaatttagggcacttggtgggagcttcatcacctctacagactcctccacggactcaggccctgatcagacaggccttcccggctcctatgtagggccatagggccgagtcctgggctgtcttgtttgtggaggtcaagtggagacagacaaaacgtgaccacgcaatccctttacggcaaagctctcccagaacagacctagctcctaaacctgtttgaacaaaaacagttgtgaaacagatcatgagaaaaggaacctggtgtccctataggaaagggtaggaacctgggggaagtgttttagatcagtgcatgtgacatcgacagcagtgttacagaaaggacaggttgaggctggagtgccagctgggcacccaagccctaggttctagccccagcccaagaagaaaaacttagccctccaggtaactcaaytttgaaaactcttaccgggtagctgagggtgcccttgsatcctcttgcctctatcttccaagtactggcattatagcaagttatcccagtacatacacaccacacccacacactcaacacacacactgacacacacatacacactttttgagacaagctcttatgcagcccaagcttgcttgctttgctgtggagggcgatccccctgcctccgctccgcagcgaatgagcacttacatgcaccaggatggctccatgatagccacatctacattctacccaccctgaggcttgttttgagactgcttcctttcttcattaattcagtcatcattacatttttattttgattatacagtgagttgacacatgctacaattcctgtgtggaggtcagaggacgactttgctggagttggtgctctccttccaccgtgtgggacccagggatttgaattgggtcatcaagagttgggacaggaacctttacctgctgggctatctcacccctctcttgttttgaggctgcatctcgctacacaggcctggatggtcctggaactcactgtgcaagccaggctggcttcaggcttgcagctgccctcgggtcccccctcaccccccaagtgctgggactgtaactgtaggattgtaccatgcctgcctgtagttctttaaatagaatatatcagtagccttaaaagtgagagatcctattcaacagtgcctgagaattcctaaatgtgttgtgctagctattatataaacacggagaagcatagggttgttcaatttaaggggtgaaaagcatagcttgggggtacaaaatggacccctcttttctatcctagcactataaacaaaccattttctagacattaccggcaaagcatgatacaacgatttgggaaatagagtctttttttttcttttttcttttttgagacatggtctcatgtaccccaagacaacctcagacactaagatgacctgatgactttactgcctctacctccaatgtgctagggttccaggtgtgcaccaccaaagccagtttaagtgatgctgtgggtcaaacccagggcttcgtgcaagctaggcacgtgctccaccgagtcaacatgccctgcccaggtctggtctaatttcttgctgaacagtggggcactgagatccagaaagggcaagcacttgggaagagcacacagtgagacctcgggcttccagatatcagagttccttcccctgaggctggggtgacccacaattccatcccgagaagcagaaacccagttaaaaggatcattagccacctgaacgtggaccactgtgtctgcccccttaggcattaagtgggagcctggaaggtgagcaggttggccaatatcttttacaaaagccaaagcgtggttgtgtatagggggaggatggggctcggtggtaatctggcttgtcccaaaccggtgtggtccagcctgggggagatgggtaggagccagaacgtggggacagagaagcccaatgaatgggccttgaaaatctgctgccctcttgcacagggccttagctcagggtcgtgctgcagctggtcctgtctgagcccactgctgggcaggcctggtgggcatcatggcggggaggggcagcgcttctctagctctgagatgctggggacgagactgaggtaggggcacacctcaaggtaactctagggtactttcctaaccgtcactgwgctkgggtggggggtgtctctgtttcaggtcttcaagggtctggaacaaatgtgggggtccatgagaaagactcagggagggattccccgccccaccccccacccccgcccccggcacaggctaagccgctatcttscttcccctggcaagattacttctcaggccatagaaacatgaaagctcccacgcaaggcgtggatcccccgggctgcaggaattcgatatcaagcttatcgataccgtcgacctcgagggggggcccggat

SEQ ID NO: 6 - DNA, 1867 bp, Homo sapiens
  CDS (144)..(1775)
  variation (1065)..(1067): y=pyrimidine (YCT encodes CCT (aminoacid proline) or TCT (aminoacid serine))
tagaagttatccccacaagacaacactgtccccttgtcctgcagttttcagttcaagaatcctaatgtacctggctagctggtggtgagtaggggctttggggccaacttggtgggctccccaaggaaacccctttgaaaccaatggatgcattcacgggctcgggtctcaagaggaagtttgatgatgtggatgtgggctcatcagtttccaactcagatgatgagatctccagcagtgatagtgctgacagctgcgacagcctcaatcctcctaccactgccagcttcacacccacatccatcctgaagcggcagaagcagctgcggaggaagaatgtacgctttgaccaggtgactgtatactactttgcccggcgccaaggttttaccagtgtgcccagccagggtggtagctctctgggcatggcccagcggcataactctgtacggagctatacactctgtgagtttgcccaggaacaggaggtgaaccatcgagagattctgcgtgagcacctgaaggaagagaaactccatgccaagaaaatgaagctgaccaagaatgggacagtggagtcggtggaggctgatggcctgacgctggatgatgtgtcagatgaagatattgatgtggaaaatgtggaggtggatgattacttcttcctgcagcctctgcccaccaaacggcgacgggccctgctgagggcttctggggtccaccgtattgatgctgaagagaagcaagaacttcgagccatccgcctgtcacgggaagaatgtggttgtgactgccgactgtattgtgacccagaagcgtgtgcctgcagccaggctgggattaaatgccaggtggatcgcatgtcctttccatgtggctgctcccgggatggctgtgggaacatggcaggacgcattgaatttaatccaatccgggtccggactcattacctccacaccattatgaagctggagctggagagcaagcggcaggtgagccgcccagcagccccagatgaggagccctccccgactgccagttgcagcctgacaggagcacagggcyctgagacccaggacttccaggagttcattgctgagaatgagacagcagygatgcacctgcagagtgcagaggaactggagcggctcaaggcagaagaagattccagcggctctagtgccagcctggactcgagcatcgagagcctgggtgtgtgcatcctagaggagcctctggctgtccccgaagagctgtgcccaggccttacagcccccattctcatccaggctcagctgcccccaggctcctctgtcctgtgttttaccgagaactcagaccacccaactgcctcaacggtgaacagcccatcctacttgaacagtgggcccctggtctattatcaagtggagcagaggccagtcttgggagtgaaaggagagcctggtacggaagaaggctcagcctctttcccaaaggagaaggatctgaatgtcttctctctccctgttacctcactcgtggcttgtagctccacagacccagctgccctctgtaaatcagaggtggggaaaacacccaccctagaagctctattgcccgaagattgtaaccctgaggagcctgaaaatgaagacttccacccttcctggtccccctcaagcctccccttccgcacggacaatgaagagggctgtgggatggtgaagacctcccagcagaatgaggatcggccccctgaagattcttccttagaactccctctggcagtgtgacaggcgctagaggtcctgcctcttacccattctctatttattcccttattttatctaacaccatttcaaaacaaaactgtagaagcaaaaaa

SEQ ID NO: 7 - protein, 543 aa, Homo sapiens
  Unsure (308)..(308): Xaa = Pro or Ser
MDAFTGSGLKRKFDDVDVGSSVSNSDDEISSSDSADSCDSLNPPTTASFTPTSILKRQKQLRRKNVRFDQVTVYYFARRQGFTSVPSQGGSSLGMAQRHNSVRSYTLCEFAQEQEVNHREILREHLKEEKLHAKKMKLTKNGTVESVEADGLTLDDVSDEDIDVENVEVDDYFFLQPLPTKRRRALLRASGVHRIDAEEKQELRAIRLSREECGCDCRLYCDPEACACSQAGIKCQVDRMSFPCGCSRDGCGNMAGRIEFNPIRVRTHYLHTIMKLELESKRQVSRPAAPDEEPSPTASCSLTGAQGXETQDFQEFIAENETAXMHLQSAEELERLKAEEDSSGSSASLDSSIESLGVCILEEPLAVPEELCPGLTAPILIQAQLPPGSSVLCFTENSDHPTASTVNSPSYLNSGPLVYYQVEQRPVLGVKGEPGTEEGSASFPKEKDLNVFSLPVTSLVACSSTDPAALCKSEVGKTPTLEALLPEDCNPEEPENEDFHPSWSPSSLPFRTDNEEGCGMVKTSQQNEDRPPEDSSLELPLAV

SEQ ID NO: 8 - DNA, 2408 bp, Rattus norvegicus
  CDS (299)..(1906)
tgcggccgccggttctgggctttgctgcccggggagccggagtgcagagccgcccgccccccgccgcctgccggccggcccacacaccctgcagccccgagatccacgcgacgggcccgggagccggcacgcctcgatctcccctccttgcacacggactgaataggcagctagggcggcggagaccgtgacgggagaagcggctgcggaggggccgagtcctcatgctcctggctagctggtggtgagcagcttggggccatcgtggttggctccccaaggagacctttggaagccaatggatgcgttctcgggctcaggcctcaagaggaaattcgatgatgtggacgtgggctcgtcagtctccaactcggatgatgagatgtccagcagcgacagtgcagacagctgcgacagccttaaccctcctacgactgccagcttcacaccaacatccatcctgaagcgacagaagcccctgcggaggaagaatgtccgctttgaccaggtgactgtgtactacttcgcccggcgacagggtttcaccagcgtgcccagccagggtggaagctctctggggatggcccaacgccacaactctgtgcgcagttacaccctttgtgagtttgcacaagagcaagaggtgaaccacagagagattcttcgtgagcacctgaaggaggagaaacttcatgccaagaaaatgaagctgacgaagaacgggacggtggagtccgtggaggccgacggcttgaccctcgacgacgtttcagacgaagacatcgatgtggagaacgtggaggtggatgactacttcttcctccagcccctgcccaccaaacgacggcgggccctgctgagggcttccggggtccaccgcatcgatgctgaggagaaacaggagctcagagccatccgcttgtctcgggaagagtgtggctgtgactgtcggctctactgtgacccagaagcctgtgcctgtagccaggctgggattaaatgccaggtggatcgaatgtcctttccttgcggctgctccagggatggctgtgggaacatggcggggcggattgagtttaatccgatccgagtgcggactcactacctccacaccatcatgaagctggagctggagagcaaaaggcaggtgagtcgcccggcggccgctgaggatgagcccctgccgggagcgcagagctcggagacacaggacttccaggagttcatcgctgagaacgagaccgcagtgatgcatctgcagagtgcagaggaactggagcggctcaaggccgaggaggactcgagtggctcgagcgctagcctggactccagcatggagagcctgggcgtgtgcatcctggaggagcccctggctgttccccaggagctgtgtccaggccttgcagcccccattctcatccaggctcagctgcccccaggttcctccgtcctgtgttttactgagaactcagaacacccagccgcctccccgatgagcagcccatcctacttgaacagtggacctctggtgtactaccaggtagagcagaggccggtcgtgggagtgaaagcagagtctggttcagaagaaggcacagcctccttccccaaggagaaagatctgagtgtcttctccctccctgttacctcactggtggcttgcagcccttcagactccgctgctctctgtaaaccagaagtggggaaagcacccagtctcaacaaactcttgcctgacgactgtaaccgtaaggagcctgaacgcgaagacttccacccgtcttggtctccctcaagcctacccttccgaacagacaataaagaaggctgtggagtacagaactcccagcagagtgaggaccggacccctgaagactcagccctagaactccctctggcggtgtgactgagatacattgtccattctctatttattatctaatgccatttcaaaacaggactgctgctcccaggttgttttgttggaagtcttaggaaagtgggtaggaaaggttagtacatgtgttttctgagagcgcatacccaggagaccagccccagctgcagggtccgtcaccatgctctctgggccaccagaacaaaggtgtcgatcacggagcgagctcggcagctcaagcggctctttatgccgagtgccagacccaagcattgagcagctccctgctccaccctggctcctcacatcgcggcgtcttgtagcagccacactagtgggaaggccactcagctgagcctacaatggaggggagatcccagaagagcctctgcttctgtacaaaactcccaggggctcagaatgtttgttctgccttgtccccgcccctttaactggtaataagtaaatttccaacgtgtttggtgttccctgaaaaaaaaaaaaaaaaaaa

SEQ ID NO: 9 - protein, 535 aa, Rattus norvegicus
MDAFSGSGLKRKFDDVDVGSSVSNSDDEMSSSDSADSCDSLNPPTTASFTPTSILKRQKPLRRKNVRFDQVTVYYFARRQGFTSVPSQGGSSLGMAQRHNSVRSYTLCEFAQEQEVNHREILREHLKEEKLHAKKMKLTKNGTVESVEADGLTLDDVSDEDIDVENVEVDDYFFLQPLPTKRRRALLRASGVHRIDAEEKQELRAIRLSREECGCDCRLYCDPEACACSQAGIKCQVDRMSFPCGCSRDGCGNMAGRIEFNPIRVRTHYLHTIMKLELESKRQVSRPAAAEDEPLPGAQSSETQDFQEFIAENETAVMHLQSAEELERLKAEEDSSGSSASLDSSMESLGVCILEEPLAVPQELCPGLAAPILIQAQLPPGSSVLCFTENSEHPAASPMSSPSYLNSGPLVYYQVEQRPVVGVKAESGSEEGTASFPKEKDLSVFSLPVTSLVACSPSDSAALCKPEVGKAPSLNKLLPDDCNRKEPEREDFHPSWSPSSLPFRTDNKEGCGVQNSQQSEDRTPEDSALELPLAV

SEQ ID NO: 10 - DNA, 2997 bp, Rattus norvegicus
  CDS (218)..(1963)
tgcggccgccggggccggggctgagccagtctctcccgccgccgccggacgcgcagacctgggcaggctgcaccgacggccgcctggccgagcgcactgcaggtcgctgcgcgcgctgcgaccccggggcccggacgcgagtggctgcggtgtcctgggcgagcactgctagtttaggccgtctgtcctcagctgcttggaacccctacatcccaccatggctgggatacagaagaggaagtttgaccagctggaagaggacgactgcagctcctcctccttgtcctctggcgatctctctccctctcctcccagctcttctgcctcccctgcctggacctctgaggaggagggactgggtgatcagccaccccagcctgatcaggactccagtggcatccagagtttaacgcccccatccatcctgaagcgggctcctcgggagcgtccgggtcacgtggccttcgatggcatcactgtctactatttcccgcggtgccagggattcaccagtgtgcccagccatggtggctgtaccctgggcatggcttctcgtcatagcacctgccgcctcttctccttagccgagtttaaacaggagcagttccgggctcggcgtgagaagctccgtcggcgtttaaaggaggagaagctagagatgctgaaatggaagctttcagtgtccggagttccggaggcaggggcagacgtgccgctcacagtggacgccatcgatgacgcttctgtagaggaggacttggcagtggccgtggcaggtggccgcctggaggaagcgaatttcctacagccctatccacctcggcagcgacgggccctacttcgcgcttccggtgttcgaaggattgaccgagaggagaagcacgagctgcaggcgctacgccaatcccgggaggattgtggttgtcactgtgatggcgtctgtgaccctgagacctgcagttgcatcctggcgggcattaaatgccagatggatcacacgtccttcccctgtggctgctgcagcgagggctgtgagaacccccatggtcgagtggaattcaatcaggcgagagttcagacacacttcatccacacgctcacccgcctgcagatggagcagggtgcggagagtttgggggacccggagtcccccatggaggacgttcctgtcgaacaaaccgtggtttccccctttcctccttccaaacccactatgagcaatgacctgggggacagcagctgtggcagcgacatgacagactcttccacgacctactcctctggcggcagtggcagccgcagcgaggctccgaaccatcttgcccaccccagcctgccaggttccagcttccggtctggcatagatgaagacagcctggaacagatcctgaatttcagtgactctgacctcggtattgaggaagaagaggaggagggagggagtgtgggcaacttggataacctcagctgttttcatttggctgacatctttggtaccggtgaccccggcagcctggctagctggacacacagccagtttggctctagccttccatcgggcatcctagatgagaatgccaacctggacgccagctgcttcctaagcagcggactcgaagggttgagagaaggtagcctccccagcagttctgggtcccctgagggggaagccgcccagagcagctccttggacctcagtttatcctcctgtgactcctttgagcttctccaatctctgccagattatagtctggggcctcactatacttcccgaagggtatctggcagcctggacagccttgagaccttccacccttcgcccagcttctctccaccgagggatgccagcttcctggattctctcataggcctgtctgagccggttacagatgtcctggcgccccttctggagagccagtttgaggacactgctgtggtgcctttggaccctgtgcctgtgtaaggaytgagatgactttttcctgccctgagaccctgttgctgctttttatgtgatcttggtgtcccccaaggtctgtgtatgtaacggtctcccgtgggctggttctgcccccgtgccatgtgggcaatcctctatttttacagtaacactctagatttatttatttttttatgtttttctgtactgaagggagggtgggaagggtatccctctttcaatgcctggcctctatgtccaaacagaggtctcccacctcctactgtatgcctggaggaggaaggggcggggttcacatcccctctttctgtactgtaaaatgctccttggtccaaagacagctgaaaagcaggccttagggtttcctgtggaccgtgggagctaggtcttctggactgtgaagatgtaatttatttctgtaatttatttggggactgagacagcagtggttgggcctctctggcaggtgggcggtgttgaggcaaagtcttcggtgtcccccgccggtctgggcttcggtgtggcgtgtaggttcgagctgagcagacggaggctgtgcttgaccatcggtgatcaaaactccctctgccccctgcccagacgctctaacatgccctctgtccatttccctctccccaaggccatgggttataaaggccctatgtaggatggggagccagaggccctaagacatgaagcacaccccagatcactgtctctagcctttctgggcactgaatccatcctgacccaccacacaccccccggccagttggcaagaaagaggtggctcttgggggcttttatgcccttcattagctgatgttggattttatatgcatttttatattgtctctaarwrwcagaactataatttattcatttctctgtgtgtgtgtgtgccaagaaacgcaggctctgggcctgcctccttgcccaggaggccttgccagcctgtgtgcttgtgagaacacattgtacctgagctgacaggtaccaataaagacactctatttttaaaaaaaaaaaaaaaaaaa

SEQ ID NO: 11 - protein, 581 aa, Rattus norvegicus
MAGIQKRKFDQLEEDDCSSSSLSSGDLSPSPPSSSASPAWTSEEEGLGDQPPQPDQDSSGIQSLTPPSILKRAPRERPGHVAFDGITVYYFPRCQGFTSVPSHGGCTLGMASRHSTCRLFSLAEFKQEQFRARREKLRRRLKEEKLEMLKWKLSVSGVPEAGADVPLTVDAIDDASVEEDLAVAVAGGRLEEANFLQPYPPRQRRALLRASGVRRIDREEKHELQALRQSREDCGCHCDGVCDPETCSCILAGIKCQMDHTSFPCGCCSEGCENPHGRVEFNQARVQTHFIHTLTRLQMEQGAESLGDPESPMEDVPVEQTVVSPFPPSKPTMSNDLGDSSCGSDMTDSSTTYSSGGSGSRSEAPNHLAHPSLPGSSFRSGIDEDSLEQILNFSDSDLGIEEEEEEGGSVGNLDNLSCFHLADIFGTGDPGSLASWTHSQFGSSLPSGILDENANLDASCFLSSGLEGLREGSLPSSSGSPEGEAAQSSSLDLSLSSCDSFELLQSLPDYSLGPHYTSRRVSGSLDSLETFHPSPSFSPPRDASFLDSLIGLSEPVTDVLAPLLESQFEDTAVVPLDPVPV

SEQ ID NO: 12 - DNA, 1392 bp, Rattus norvegicus
aggaggctgcaagagggcgggaggcaggagagggcccggagctggtgggccggagcggcggcgccgccatgtccgacagcgagaagctcaacctggattccatcatcgggcgcctgctggaagtgcagggctcacggcctggaaagaatgtgcagctgacagagaacgagatccgtggtctttgcctcaaatcccgggagattttcctgagccagcctattcttctggagcttgaggcgcctctcaagatctgtggtgacatccatggccagtactatgaccttctacggctgttcgagtatggtggcttccctccagagagcaactacctcttcttgggggactatgtggatcggggaaagcagtctttggagaccatctgcttgttgctggcctataagatcaaatacccggagaatttctttctactacgtgggaaccatgagtgtgccagcatcaaccgcatctatggcttctatgatgaatgcaagagacgatacaacatcaaactgtggaagactttcaccgactgcttcaactgcctgcccattgcagccattgtagatgagaagatcttctgctgccacggaggcctgtctccagacttgcaatccatggagcagattagacgtattatgcggcccacggacgtgcctgaccagggcctgttgtgtgatctgctgtggtctgaccctgacaaggatgttcaaggctggggcgagaatgaccgtggagtctcctttacctttggggccgaggttgtagccaagttcctgcacaagcatgatttggacctcatctgcagagcacatcaggttgtagaagatggctatgagttctttgccaagaggcagctggtgacactcttctcagctcccaactactgtggcgagtttgacaacgctggcgccatgatgagtgtggacgagacactcatgtgttccttccagatcctcaagcccgctgataagaataaggggaagtatgggcagttcagtggcctgaaccccggaggccgtcccatcactccaccccgcaattctgccaaagccaagaaatagcctccatgtgctgccctctgccccagatgacggattatttgtacagaaatcatgctgccatgggtcacactggcctctcaggcccacccatcatggggaacacagcgttaagtgtctttcctttattttttaaagaatcaatagcagcatctaattccccagggctccctcccaccagcacctgtggtggctgcaagtggaatcctggggccaaggctgcagctcagggcaatggcagaccagattgtgggtctccagccttgcatggctggcagccagatcctggggcaacccatctggtctcttgaataaaggtcaaagctggattctc

SEQ ID NO: 13 - DNA, 597 bp, Rattus norvegicus
gtcgacccacgcgtccgggattgggagggaaggagctctcagttgcagccatgccgaatcacggtcgctgatcagggtccctcacggggctgctgggagaacccagagagcagcgccagttcccgggtgcatgcacagtttggtggctactaaccctgccgggcctcccacaatggagggcgaaagcgtcaagccgagccctcagcccacagagcaggccggggatggtgagaagaaccggaggatgatcactgtcaaccctgcccacatggggaaggccttcaaggtcatgaatgagctgcgaagtaagcggctgctgtgtgacgtgatgatcgtggcagaggacgtggaggtagaagcccaccgcgtggtcctggcagcctgcagcccttacttctgtgcgatgttcacaggtgacatgtctgagagcaaagccaagaagattgaaatcaaggacgtggacggacagaccctcagcaagctcatcgactacatctacaccgcggagatagaggtgacggaggagaacgtgcaggtcctgcttccagcagccagcttgctgcagctcatggacgtccgtagaactgctgtgacttt

SEQ ID NO: 14 - DNA, 1547 bp, Rattus norvegicus
gaattcggcacgagggaggcacccgaagcagccctggcggaccaaggagcgatggcagcggccgatatagctcgccaggtgggcgaagattgtcgaactgtccccctggctggacatgtggggttcgacagcttgcctgaccagctggtgaataagtcagtcagccagggcttctgcttcaacatcctgtgcgtgggggagacaggtttgggcaagtccacgctcatggataccctgtttaacaccaagtttgaaggggagccagcgacccacacacagccaggggttcagctccagtccaatacctacgacctccaggagagcaacgtggggttgaaactcacaattgtgagcactgtaggctttggggatcagatcaacaaggaggacagctacaagcccatcgtggaattcattgatgctcagtttgaggcctacctacaagaggagctgaagatccgaagagtactgtactcctaccatgactcccgaatccatgtctgcttgtatttcattgccccgacaggacattcgcttaagtctctggacctagtgaccatgaagaagctggatagtaaggtgaacatcattcccgttattgccaagtcagatgccatttctaagagtgagctggcaaagttcaagatcaaaatcaccagcgaacttgtgagcaatggagtccagatctatcagttccccacagacgatgagtcggtgagcgagatcaatggaaccatgaatgcccacctgccgtttgctgttgttggcagcacagaagaagtgaagataggcaacaagatgatgcgggctcggcagtatccttgggggactgtgcaggttgaaaacgaggctcactgcgactttgtgaagctgcgggagatgctgattcgggtcaacatggaggatctgcgggagcagacccacgcccggcactatgaattgtaccgccgctgtaagctggaggagatgggcttcaaggacaccgaccctgacagcaaaccattcagtttgcaggagacatatgaggcaaagagaaatgagttccttggggagctccagaagaaagaagaggagatgaggcagatgtttgtccagagagtcaaagagaaagaagcggagctcaaggaggctgaaaaagagctgcatgagaaatttgaccgtctgaagaaactgcaccaggaagagaagaagaagctggaggataagaagaaatgtctggatgaggaaatgaacgccttcaagcagagaaaggcggcacgtgagctgctccagtctcagggctctcaggctgggggctcacagactctgaaaagggacaaagagaagaagaactttttttaacgttgtcttcagcagctgtactaagtctaaaggagaagactgccattatagaagagttagttagggctccatactgtctcaagcaagaaaccaaccaatccccctcccctgcaactgcgcacatatacaccacactcaacaagtgtacatgtgtccctgtgtccaggcaagaagctctcttctgacttacatggtgtttcacatgg

SEQ ID NO: 15 - DNA, 21 bp, Artificial Sequence Sequence
  PCR primer 1 for amplifying L100
gaccatcggtgatcaaaactc

SEQ ID NO: 16 - DNA, 22 bp, Artificial Sequence
  PCR primer 2 for amplifying L100
acatcagctaatgaagggcata

SEQ ID NO: 17 - DNA, 23 bp, Artificial Sequence
  PCR primer 1 for amplifying S26
aagtttgtcattcggaacattgt

SEQ ID NO: 18 - DNA, 21 bp, Artificial Sequence
  PCR primer 2 for amplifying S26
cacctctttacatgggctttg

SEQ ID NO: 19 - DNA, 24 bp, Artificial Sequence
  L100 sense primer
gaagaggaagtttgaccagctgga

SEQ ID NO: 20 - DNA, 24 bp, Artificial Sequence
  L100 antisense primer
agtcctcctctacagaagcgtcat

SEQ ID NO: 21 - protein, 11 aa, Artificial Sequence
  antigenic tag Myc
MEQKLISEEDL

SEQ ID NO: 22 - protein, 9 aa, Artificial Sequence
  antigenic tag Flag
MDYKDDDDK

SEQ ID NO: 23 - DNA, 34 bp, Artificial Sequence
  PCR primer L100-5′-Y2H-407
acagcgacgtcgacggagggagtgtgggcaactt

SEQ ID NO: 24 - DNA, 34 bp, Artificial Sequence
  PCR primer L100-3′Y2H-581
atagtttagcggccgcttacacaggcacagggtc

SEQ ID NO: 25 - DNA, 32 bp, Artificial Sequence
  PCR primer L100 5′2-Y2H
caaacgcgtcgaccgctgggatacagaagaag

SEQ ID NO: 26 - DNA, 37 bp, Artificial Sequence
  PCR primer L100-3′-315-Y2H
atagtttagcggccgcttagtcctccatgggggactc

SEQ ID NO: 27 - DNA, 21 bp, Artificial Sequence
  PCR primer pPC86a
gtataacgcgtttggaatcac

SEQ ID NO: 28 - DNA, 22 bp, Artificial Sequence
  PCR primer pPC86b
gtaaatttctggcaaggtagac

SEQ ID NO: 29 - protein, 114 aa, Rattus norvegicus
NPAHMGKAFKVMNELRSKRLLCDVMIVAEDVEVEAHRVVLAACSPYFCAMFTGDMSESKAKKIEIKDVDGQTLSKLIDYIYTAEIEVTEENVQVLLPAASLLQLMDVRRTAVTF

SEQ ID NO: 30 - protein, 114 aa, Drosophila melanogaster
NEQHTARSFDAMNEMRKQKQLCDVILVADDVEIHAHRMVLASCSPYFYAMFTSFEESRQFARITLQSVDARALELLIDYVYTATVEVNEDNVQVLLTAANLLQLTDVRDACCDF

SEQ ID NO: 31 - protein, 101 aa, Mus musculus
PQHSQRVLEQLNQQRQLGLLCDCTFVVDGVDFKAHKAVLAACSEYFKMLFVDQKDVVHLDISNAAGLGQVLEFMYTAKLSLSPENVDDVLAVASFLQMQDI

(19) United States
(12) Patent Application Publication
(10) Pub. No.: US 2005/0147967 A1
(43) Pub. Date: Jul. 7, 2005

(54) Novel neuronally expressed protein and use thereof

(76) Inventors: Götz Bernhard, Heidelberg (DE); Brigitta Kammandel, Heidelberg (DE); Rohini Kuner, Heidelberg (DE); Sigrid Scheek, Dossenheim (DE); Holger Hiemisch, Heidelberg (DE)

Correspondence Address: BIRCH STEWART KOLASCH & BIRCH, PO BOX 747, FALLS CHURCH, VA, 22040-0747, US

(21) Appl. No.: 10/257,763

(22) PCT Filed: Apr. 17, 2001
(86) PCT No.: PCT/EP01/04311

(30) Foreign Application Priority Data
Apr. 20, 2000 (DE) 100 19 901.1

(51) Int. Cl. C12Q 1/68; G01N 33/574; C07H 21/04; A61K 48/00; C12P 21/02; A61K 39/395; C12N 9/64

(52) U.S. Cl. 435/6; 435/7.23; 435/69.1; 435/226; 435/320.1; 435/325; 514/44; 530/388.22; 536/23.2; 424/146.1

(57) ABSTRACT

The invention relates to novel specifically expressed proteins, and to nucleic acid sequences or recombinant nucleic acid constructs encoding said proteins. The invention further relates to host organisms or transgenic animals containing said nucleic acid sequences or recombinant nucleic acid constructs, and to monoclonal or polyclonal antibodies directed to the isolated proteins. The invention also relates to a method for finding substances having a specific binding affinity to the inventive proteins, and to a method for the qualitative or quantitative analysis of the inventive nucleic acid sequences or inventive proteins. The invention further relates to the use of the nucleic acid sequences and proteins.

The present invention relates to novel, specifically expressed proteins and to nucleic acid sequences or recombinant nucleic acid constructs which encode the proteins.

The invention also relates to host organisms or transgenic animals which comprise the nucleic acid sequences or recombinant nucleic acid constructs and to monoclonal or polyclonal antibodies which are directed against the isolated proteins.

The invention furthermore relates to a method for finding substances possessing specific binding affinity for the proteins according to the invention and to a method for qualitatively or quantitatively detecting the nucleic acid sequences according to the invention or the proteins according to the invention. In addition, the invention relates to the use of the nucleic acid sequences and proteins.

The nucleic acids according to the invention form a novel gene family which belongs to what are termed the immediate early genes (=IEGs).

All the previously described cloned IEG genes are expressed neuronally [Brakeman et al., (1997), Nature 386: 284-288; Kaufmann et al., (1994), Int. J. Dev. Neurosci. 12: 263-271; Kato et al., (1998) J. Biol. Chem. 273: 23969-23975; Lyford et al., (1995) Neuron 14: 433-445; Tsui et al., (1996) J. Neurosci. 16: 2463-2478; Kaufmann et al., (1997), Brain Dev. 19: 25-34; Wallace et al., (1998) J. Neurosci. 18: 26-35; Steward et al., (1998), Neuron 21: 741-751; O'Brien et al., (1999), Neuron 23: 309-323].

A stimulus rapidly increases their expression. For example, it has been shown that IEG Homer 1A is a truncated variant of a member of a relatively large gene family and that induction of this variant leads to the (dominant-negative) interruption of the signal transmission which the other members of the gene family mediate between extracellular receptors and internal calcium stores [Tu et al., (1998), Neuron 21: 717-726; 73; Xiao et al., (1998), Neuron 21: 707-716; Yuguchi et al., (1997), J. Cereb. Blood Flow Metab. 17: 745-752]. Consequently, an external stimulus (e.g. a convulsion) leads to direct changes in important second messenger systems.

Another example of the important role played by the neuronally expressed IEGs in the hippocampus is what is termed Arc. This gene is likewise induced to express its mRNA in pyramidal cells of the hippocampal sub-regions CA1 and CA3 after a convulsion has been elicited. It has been shown that expression of the Arc mRNA is induced in the CA1 area by simply placing the rat in a new environment. Since the pattern of the neurons which were induced was specific for the particular environment in which the rat was located, it was possible to demonstrate that induction of Arc mRNA expression correlates with processes of neuronal information storage in the hippocampus [Guzowski et al., (1999), Nat. Neurosci. 2: 1120-1124]. The so-called IEGs appear to be important intracellular regulatory points. They play an important role in the development of organisms and in pathological processes within the organisms or within the individual cell.

The object therefore was to make available further IEGs which can be used as intervention points when treating diseases. This object was achieved by means of an isolated nucleic acid sequence which encodes a polypeptide possessing apoptase activity and which is selected from the group consisting of:
a) a nucleic acid sequence having the sequence depicted in SEQ ID NO: 1, SEQ ID NO: 3, SEQ ID NO: 5, SEQ ID NO: 6 or SEQ ID NO: 8,
b) nucleic acid sequences which, as a result of the degeneracy of the genetic code, are derived from the nucleic acid sequence depicted in SEQ ID NO: 1, SEQ ID NO: 3, SEQ ID NO: 5, SEQ ID NO: 6 or SEQ ID NO: 8,
c) derivatives of the nucleic acid sequence depicted in SEQ ID NO: 1, SEQ ID NO: 3, SEQ ID NO: 6 or SEQ ID NO: 8 which encode polypeptides having the amino acid sequences depicted in SEQ ID NO: 2, SEQ ID NO: 4, SEQ ID NO: 7 or SEQ ID NO: 9 and which exhibit at least 90% homology on the amino acid level without the biological activity of the polypeptides being significantly reduced.

These nucleic acids can be found in eukaryotic and prokaryotic organisms, advantageously Mammalia such as Homo sapiens, Rattus norvegicus or Mus musculus. These nucleic acids encode the amino acid sequences SEQ ID NO: 2 (Homo sapiens), SEQ ID NO: 4 (Mus musculus), SEQ ID NO: 7 (Homo sapiens) and SEQ ID NO: 9 (Rattus norvegicus). According to the genomic data, human L100 (SEQ ID NO: 1) is located on chromosome 3 while the human L100 homolog (SEQ ID NO: 6) is located on chromosome 12.

A first cDNA clone belonging to this gene family was isolated from the rat (WO 99/40225). The cDNA clone which was obtained in this way encodes a protein which was termed the L100 protein. The sequence of the rat L100 is reproduced in SEQ ID NO: 10. SEQ ID NO: 11 is the appurtenant amino acid sequence. The representatives of this gene family appear to belong to what are termed the neuronal immediate early genes (=IEGs).

A gene is termed an IEG if it fulfils three conditions:
a) its mRNA must be expressed at a low level in resting or unstimulated cells,
b) its mRNA can also be expressed in the absence of de novo protein synthesis
c) the transcription of the gene is activated after suitable stimulation.

IEGs are frequently subdivided into 3 classes based on the kinetics of the accumulation of the mRNA.
a) Class I IEGs can frequently not be detected in resting or unstimulated cells. The maximum mRNA concentration is reached about 30-60 minutes after stimulation. After from about 1.5 to 2 hours, the concentration of the mRNA returns once again to the basal values (examples are c-fos, c-jun and zif268).
b) Class II IEGs reach maximum mRNA concentrations 2 hours after stimulation. The basal values are reached once again after about 8 hours (examples of these IEGs are Narp, c-myc and GLUT1).
c) Class 3 genes are induced very rapidly and their mRNAs accumulate over many hours. These mRNAs exhibit a long half-life (e.g. fibronectin).

Based on these criteria, the proteins according to the invention and the nucleic acids encoding the proteins L100 and L100 homologs can be classified as being class I IEGs. The mouse L100 cDNA exhibits a high degree of homology in mouse Fanconi's anemia complementation group A fragment in the 5′-untranslated region (AF 208 119). No agreement is found at the protein level.

Derivatives of the nucleic acid sequences according to the invention having the sequence SEQ ID NO: 1, SEQ ID NO: 3, SEQ ID NO: 5, SEQ ID NO: 6 or SEQ ID NO: 8 are, for example, to be understood as meaning allelic variants which exhibit at least 90% homology on the deduced amino acid level, preferably at least 92% homology, very particularly preferably at least 95% homology, over the entire sequence region. The homologies can advantageously be higher over constituent regions of the sequences. Allelic variants comprise, in particular, functional variants which can be obtained by the deletion, insertion or substitution of nucleotides from the sequence depicted in SEQ ID NO: 1, SEQ ID NO: 3, SEQ ID NO: 5, SEQ ID NO: 6 or SEQ ID NO: 8, in connection with which the biological activity of the derived, synthesized proteins should not be significantly reduced. Proteins having a biological activity which is not significantly reduced are to be understood as being proteins which exhibit a biological activity of at least 20%, preferably 50%, particularly preferably 75%, very particularly preferably 90%, calculated in accordance with the algorithm of Pearson and Lipman, Proc. Natl. Acad. Sci (USA), (1988), 85(8), 2444-2448, or alignments were carried out in accordance with ALIGN calculates of Myers and Miller CABIOS (1989), 4: 11-17. The invention consequently also relates to amino acid sequences which are encoded by the group of nucleic acid sequences depicted above. The invention advantageously relates to amino acid sequences which are encoded by the sequence SEQ ID NO: 1, SEQ ID NO: 3, SEQ ID NO: 5, SEQ ID NO: 6 or SEQ ID NO: 8.

In this connection, allowances are naturally to be made for certain variations; for example, SEQ ID NO: 6 (human L100 homolog) contains 2 unresolved positions, i.e. nt 1065 and 1114. Sequence analysis gave a T for position 1065 in one lambda clone while two clones gave a C at this position; a T was sequenced 4 times at position 1114 while a D was sequenced once. The genomic entries have a T at both positions.

Functional equivalents of the sequences mentioned under (a) to (c) are to be understood as meaning nucleic acids which encode proteins which possess the biological activity of L100 and its homologs and which possess at least 50% of the activity of the sequences specified under SEQ ID NO: 2, SEQ ID NO: 4, SEQ ID NO: 7 or SEQ ID NO: 9. These functional equivalents advantageously interact with other proteins, with which they form what are termed protein complexes (=protein heteromers).

The essential biological property of the proteins according to the invention is furthermore to be understood as meaning, for example, the membrane domains and the cysteine-rich, serine-rich and glutamine-rich domains and also the nuclear localization domain. This property enables the proteins to have their specific biological effect.

The isolated protein, and its functional variants, can advantageously be isolated from the human or animal brain.

A further essential biological property is the ability to interact with proteins, that is other receptors.

The invention also relates to proteins which also possess receptor binding activity and which can be prepared from the amino acid sequence depicted in SEQ ID NO: 2, SEQ ID NO: 4, SEQ ID NO: 7 or SEQ ID NO: 9 by making selective changes. In this connection, particular amino acids can, for example, be replaced with amino acids possessing similar physicochemical properties (space-filling, basicity, hydrophobicity, etc.). For example, arginine residues are replaced with lysine residues, while valine residues are replaced with isoleucine residues or aspartic acid residues are replaced with glutamic acid residues. However, it is also possible for the order of one or more amino acids to be switched or for one or more amino acids to be added or removed, or several of these measures can be combined with one other.

Derivatives are furthermore also to be understood as meaning homologs of SEQ ID NO: 1, SEQ ID NO: 3, SEQ ID NO: 5, SEQ ID NO: 6 or SEQ ID NO: 8, for example eukaryotic homologs, truncated sequences, single-strand DNA or RNA belonging to the coding DNA sequence or to the noncoding DNA sequence. At the DNA level, homologs of SEQ ID NO: 1, SEQ ID NO: 3, SEQ ID NO: 5, SEQ ID NO: 6 or SEQ ID NO: 8 possess a homology of at least 80%, preferably of at least 85%, particularly preferably of at least 90%, and very particularly preferably of at least 95%, over the entire DNA region given in SEQ ID NO: 1, SEQ ID NO: 3, SEQ ID NO: 5, SEQ ID NO: 6 or SEQ ID NO: 8.

In addition, homologs of SEQ ID NO: 1, SEQ ID NO: 3, SEQ ID NO: 5, SEQ ID NO: 6 or SEQ ID NO: 8 are to be understood as meaning derivatives such as, for example, promoter variants. The promoters, which are located upstream of the given nucleotide sequences, can be altered by means of one or more nucleotide substitutions, or by (an) insertion(s) and/or (a) deletion(s) without, however, the functionality or the activity of the promoters being impaired. Furthermore, the activity of the promoters can be increased by altering their sequence, or the promoters can be completely replaced with more active promoters, including those from organisms belonging to a different species.

The nucleic acid sequences according to the invention SEQ ID NO: 1, SEQ ID NO: 3, SEQ ID NO: 5, SEQ ID NO: 6 or SEQ ID NO: 8 were used to carry out a blast search [BLASTN 2.0.11 (Jan.-20-2000), Altschul et al., (1997), Nucleic Acid Res., 25: 3389-3402] for the EST sequences in various nucleic acid sequence databases. In this connection, the following sequences (EMBL sequences) were found in the databases:
AI505116, AA623763, AW476299, D21599, AA370045, AA956920, AA135236, AI027708, AV272584, AI923037, AA429440, AI806476, AA305194, AI374746, AI205148, AI919283, AW251698, AW251940, AW254416, AW358288, AW347356, AW344462, AI710614, AW254235, AI713621, AI902420, AI713491, AW484184, AW426087, AI171010, AA849418, AA814410, AW313920, AI011502, AA990527, AI416381, AV168326, AV325720, AA429440, AI806476, AA305194, AI374746, AI205148, AI919283, AW251698, AW251940, AW254416, AW358288, AW347356, AW344462, AI710614, AW254235, AI713621, AI902420, AI713491, AW484184, AW426087, AI171010, AA849418, AA814410, AI011502, AA990527, AI416381, AV168326, AV325720, AW251698, AW251940, AW254416, AI710614, AW254235, AI713621, AI713491, AA305194, AA429440, AI171010, AW358288, AW347356, AA990527, AW344462, AI205148, AA849418, AI806476, AI374746, AV168326, AI011502, AV325720, AI902420, AI919283, AI416381, AW484184, AW426087, AI794507, AI027708, H16294, AA135123, AA135236, R93263, AI760726, AA961632, AA559951, AI612798, AW404376, R76656, AI923037, AA370045, AI864137, AI201837, AA635636, C01543, T46994, AW345830, AI505116, AA623763, D21599, AW477674, AW476299, AI794507, AI559002, AA589864, AA105097, AA511916, AA510012, AA274717, W44097, AA371009, AA349120, AA310845, AA310224, F12570, R11972, R69553, H08524, AA796994, R61424, R18375, AA505159, AA448038, AW408260, AI752011, AI558552, AA542671, AI404829, AI295106, AI238760, AI221701, AI103093, AI045639, AA893761, AA891212, AA212991, AA512017, AW413387, AW408302, AW379003, AW144760, AI938734, AI900542, AI900402, AI713995, AI713415, AI710191, AI502255, AA565208.

The abovementioned EST sequences are sequences which possess a certain degree of homology with parts of the sequences according to the invention but whose function is unknown.

Derivatives are also to be understood as meaning variants whose nucleotide sequence in the region of from −1 to −1000 upstream of the start codon or from 0 to 2000 base pairs downstream of the stop codon has been altered such that expression of the gene and/or expression of the proton is altered, preferably increased.

The nucleic acid sequences according to the invention can in principle be identified in, and isolated from, any organism. Advantageously, SEQ ID NO:. 1, SEQ ID NO: 3, SEQ ID NO: 5, SEQ ID NO: 6 or SEQ ID NO: 8, or its homologs, can be isolated from eukaryotic organisms, such as Caenorhabditis elegans, Drosophila melanogaster, the zebrafish, Danio rerio, or Mammalia, such as the rat, the mouse or humans. The nucleic acid sequence(s) [the plural and the singular are intended to be synonymous as far as the Application is concerned] can preferably be isolated from Mammalia.

For example, customary hybridization methods or the PCR technique can be used to isolate SEQ ID NO: 1 or its derivatives or homologs, or parts of the sequences, from Mammalia. These DNA sequences hybridize with the sequences according to the invention under standard conditions. Short oligonucleotides from the conserved regions, for example from the cysteine-rich region, which oligonucleotides can be determined, in a manner known to the skilled person, by comparing with the similar metallothioneins (=MTs), are advantageously used for the hybridization. However, it is also possible to use longer fragments of the nucleic acids according to the invention, or the complete sequences, for the hybridization. These standard conditions vary depending on the nucleic acid, i.e. oligonucleotide, longer fragment or complete sequence, employed or depending on which nucleic acid type, i.e. DNA or RNA, is used for the hybridization. Thus, the melting temperatures for DNA:DNA hybrids are, for example, approx. 100° C. lower than those of DNA:RNA hybrids of the same length.

Depending on the nucleic acid, standard conditions are to be understood, for example, as meaning temperatures of between 42 and 58° C. in an aqueous buffer solution having a concentration of from 0.1 to 5×SSC (1×SSC=0.15 M NaCl, 15 mM sodium citrate, pH 7.2) or, in addition, in the presence of 50% formamide, such as 42° C. in 5×SSC, 50% formamide. Advantageously, the hybridization conditions for DNA:DNA hybrids are 0.1×SSC at temperatures of between about 20° C. and 45° C., preferably of between about 30° C. and 45° C. For DNA:RNA hybrids, the hybridization conditions are advantageously 0.1×SSC at temperatures of between about 30° C. and 55° C., preferably of between about 45° C. and 55° C. These temperatures which are given for the hybridization are melting temperature values which are calculated, by way of example, for a nucleic acid having a length of approx. 100 nucleotides and a G+C content of 50% in the absence of formamide. The experimental conditions for the DNA hybridization are described in specialist genetics textbooks such as Sambrook et al., “Molecular Cloning”, Cold Spring Harbor Laboratory, 1989, and can be calculated, for example depending on the length of the nucleic acids, on the nature of the hybrids or the G+C content, in accordance with formulae which are known to the skilled person. The skilled person can obtain further information with regard to hybridization from the following textbooks: Ausubel et al. (eds), 1985, Current Protocols in Molecular Biology, John Wiley & Sons, New York; Hames and Higgins (eds), 1985, Nucleic Acids Hybridization: A Practical Approach, IRL Press at Oxford University Press, Oxford; Brown (ed), 1991, Essential Molecular Biology: A Practical Approach, IRL Press at Oxford University Press, Oxford.

The nucleic acid sequences according to the invention, or their fragments, can be used, under the above-mentioned hybridization conditions, for isolating a genomic sequence by way of homology screening.

The nucleic acid construct according to the invention (=expression cassette) is to be understood as meaning L100 sequences such as SEQ ID NO: 1, SEQ ID NO: 3, SEQ ID NO: 5, SEQ ID NO: 6 or SEQ ID NO: 8, and their derivatives and homologs, which have been functionally linked, advantageously for increasing gene expression, to one or more regulatory signals. For example, these regulatory sequences are sequences to which inducers or repressors bind and in this way regulate the expression of the nucleic acid. In addition to these new regulatory sequences, the natural regulation of these sequences can still be present upstream of the actual structural genes and, where appropriate, have been genetically modified such that the natural regulation has been switched off and the expression of the genes has been increased. However, the expression cassette can also be constructed more simply, i.e. no additional regulatory signals have been inserted upstream of the sequence SEQ ID NO: 1, SEQ ID NO: 3, SEQ ID NO: 5, SEQ ID NO: 6 or SEQ ID NO: 8, or its homologs, and the natural promoter, together with its regulation, has not been removed. Instead of this, the natural regulatory sequence is mutated such that there is no longer any regulation and gene expression is increased. In addition, the expression cassette can also advantageously contain one or more so-called enhancer sequences which are functionally linked to the promoter and which enable the nucleic acid sequence to be expressed at an increased level. Additional, advantageous sequences can also be inserted at the 3′ end of the DNA sequences as further regulatory elements or terminators. The nucleic acids according to the invention can be present in the construct in one or more copies. Where appropriate, the construct can contain still further markers, such as antibiotic resistances or genes complementing auxotrophies, for the purpose of selecting for the construct.

Advantageous regulatory sequences for the method according to the invention are present, for example, in promoters such as the cos, tac, trp, tet, trp-tet, lpp, lac, lpp-lac, lacI^{q}, T7, T5, T3, gal, trc, ara, SP6, λ-P_{R} or λ-P_{L} promoter, which promoters are advantageously used in Gram-negative bacteria. Further advantageous regulatory sequences are present, for example, in the Gram-positive promoters amy and SPO2, and in the yeast or fungal promoters ADC1, MFα, AC, P-60, CYC1, GAPDH, TEF, rp28 and ADH. The particularly advantageous Mammalia promoters, such as the CMV, RSV, SV40, EF-1α, CAM-kinase II, nestin, L7, BDNF, NF, MBP, NSE, β-globin, GFAP, GAP443, tyrosine hydroxylase or kainatel receptor subunit 1 promoters, should also be mentioned in this connection. It is also possible to use artificial promoters for the regulation.

For expression in a host organism, the expression cassette is advantageously inserted into a vector, such as a plasmid, a phage or another DNA which enables the genes to be optimally expressed in the host. These vectors constitute a further embodiment of the invention. pLG338, pACYC184, pBR322, pUC18, pUC19, pKC30, pRep4, pHS1, pHS2, pPLc236, pMBL24, pLG200, pUR290, pIN-III¹¹³-B1, λgt11 and pBDCI are examples of plasmids which are suitable for use in E. coli while pIJ101, pIJ364, pIJ702 and pIJ361 are examples which are suitable for use in Streptomyces, pUB110, pC194 and pBD214 are examples which are suitable for use in Bacillus, pSA77 or pAJ667 are examples which are suitable for use in Corynebacterium, pALS1, pIL2 and pBB116 are examples which are suitable for use in fungi, 2 μM, pAG-1, YEp6, YEp13 and pEMBLYe23 are examples which are suitable for use in yeasts and pLGV23, pGH1ac⁺, pBIN19, pAK2004 and pDH51 are examples which are suitable for use in plants. These plasmids represent a small selection of the possible plasmids. Examples of vectors which are advantageous for Mammalia are pcDNA3.1, pci-neo, pRc/CMV2 and pRc/RSV. Other plasmids are well known to the skilled person and can be obtained, for example, from the book Cloning Vectors (Eds. Pouwels P. H. et al. Elsevier, Amsterdam-N.Y.-Oxford, 1985, ISBN 0 444 904018).

Advantageously, the expression cassette for expressing the other genes which are present additionally contains 3′ and/or 5′ terminal regulatory sequences for increasing expression, which sequences are selected for optimal expression in dependence on the host organism selected and on the gene or genes.

These regulatory sequences are intended to enable the genes and the protein to be expressed selectively. Depending on the host organism, this can mean, for example, that the gene is only expressed or overexpressed after induction or that it is expressed and/or overexpressed immediately.

In this connection, the regulatory sequences or factors can preferably exert a positive influence on the expression of the genes which have been introduced and thereby increase this expression. Thus, the regulatory elements can advantageously be augmented on the transcription level by using strong transcription signals such as promoters and/or enhancers. However, it is also possible, in addition, to augment the translation by, for example, improving the stability of the mRNA.

A preferred embodiment is that of linking the nucleic acid sequence according to the invention to a promoter, with the promoter coming to lie 5′ upstream. Other regulatory signals, such as terminators, poly-adenylation signals and enhancers, can be used in the expression cassette.

Advantageously, the expression cassette also contains other nucleic acid sequences which encode proteins which interact with L100 or its homologs. Examples of such sequences are the sequences SEQ ID NO: 12, SEQ ID NO: 13 and SEQ ID NO: 14.

For expression in a suitable host organism, the recombinant expression cassette or the nucleic acid construct or gene construct is advantageously inserted into a host-specific vector which enables the genes to be expressed optimally in the host. As described above, vectors are well known to the skilled person and can be obtained, for example, from the book Cloning Vectors (Eds. Pouwels P. H. et al. Elsevier, Amsterdam-N.Y.-Oxford, 1985, ISBN 0 444 904018). Apart from plasmids, the vectors are also to be understood as meaning all the other vectors which are known to the skilled person, such as phages, viruses, such as SV40, CMV, baculovirus and adenovirus, transposons, IS elements, phasmids, cosmids and linear or circular DNA. These vectors can either replicate autonomously in the host organism or be replicated chromosomally.

The present nucleic acid sequence and/or the expression cassette can, in a manner known to the skilled person, be introduced into suitable systems using vectors and expressed in these systems. Advantageously, the nucleic acid sequences according to the invention, their homologs, or their functional equivalents or derivatives are introduced, as a recombinant expression cassette or as a vector, into a suitable system and expressed in this system. In this connection, customary cloning and transfection methods, which are known to the skilled person, are advantageously used for expressing said nucleic acids in various expression systems. These systems are described, for example, in Current Protocols in Molecular Biology, Ed. F. Ausubel et al., Wiley Interscience, New York 1997.

In another embodiment of the vector, the expression cassette according to the invention, or the vector containing the nucleic acids according to the invention, can also advantageously be introduced in the form of a linear DNA into the organisms and integrated into the genome of the host organism by way of heterologous or homologous recombination. This linear DNA can consist of a linearized vector, such as a plasmid, or only of the nucleic acid construct or the nucleic acids according to the invention.

In principle, all prokaryotic or eukaryotic organisms which enable the nucleic acids according to the invention, their allelic variants, or their functional equivalents or derivatives, or the recombinant nucleic acid construct, to be expressed are suitable for use as host organisms. Host organisms are to be understood as meaning, for example, bacteria, fungi, yeasts and plant or animal cells. Organisms which are preferred are bacteria, such as Escherichia coli, Streptomyces, Bacillus or Pseudomonas, eukaryotic microorganisms, such as Saccharomyces cerevisiae or Aspergillus, and higher eukaryotic cells from animals or plants, for example Sf9, HEK293 or CHO cells; eukaryotic host organisms are particularly preferred, with very particular preference being given to Mammalia or mammalian cells, such as Sf9, HEK293 or CHO cells.

If desired, the gene product can also be expressed in transgenic organisms such as transgenic animals, for example mice or sheep, or transgenic plants; transgenic animals are preferred. The transgenic organisms can be what are termed knock-out animals or plants.

The recombinant prokaryotic or eukaryotic host organisms according to the invention contain at least one nucleic acid sequence according to the invention or at least one expression cassette or at least one vector according to the invention.

The combination of the host organisms and the vectors, such as plasmids, viruses or phages, such as plasmids containing the RNA polymerase/promoter system, the phages 1 or Mu, or other temperate phages or transposons and/or other advantageous regulatory sequences, which are appropriate for the organisms, forms an expression system. The term expression systems is preferably to be understood as meaning, for example, the combination comprising mammalian cells, such as CHO cells, and vectors, such as the pcDNA3neo vector, which are suitable for mammalian cells.

In order to express heterologous genes optimally in organisms, it is advantageous to alter the nucleic acid sequences in conformity with the specific codon usage which is employed in the organism. The codon usage can easily be determined with the aid of computer analyses of other, known genes from the organism concerned.

Since IEGs can be transcriptionally activated in the absence of de novo protein synthesis, the regulatory proteins for inducing the IEGs must already be present in the unstimulated cell and be ready for an activation. It was observed that stimulating cells in the presence of cycloheximide, which is a potent inhibitor of protein synthesis, leads to IEGs being superinduced. This observation was attributed to two effects, i.e. an extended period of transcription and an increase in mRNA stability. AT-rich sequences in the 3′-untranslated region appear to play an important role for the rapid degradation of mRNAs which encode IEGs and cytokines. An AUUUA motif was identified in virtually all the IEG mRNAs which had short half-lives. The observation that inhibitors of protein synthesis stabilize IEG mRNAs can be explained by different hypotheses. On the one hand, newly synthesized or labile RNases are required for the degradation or, on the other hand, the degradation of the mRNA is directly coupled to the translation. Experimental evidence exists to support both theories. A cytosolic factor, which, after stimulation, binds c-myc mRNA and destabilizes it, and which cannot be detected during treatment with cycloheximide, has been described in the case of the c-myc gene. Numerous studies have shown that translation is a prerequisite for mRNA degradation [Rajagopalan et al., (1996), J. Biol. Chem. 271: 19871-19876]. The mRNA corresponding to the rat L100 cDNA has 5 AUUUA motifs. This makes it possible to explain the rapid regulation of the degradation of the L100 mRNA, inter alia using the above-described mechanisms.

The human and mouse nucleic acid sequences of the present invention, and their homologs, form a previously unknown gene family, which exhibits homology with metallothioneins in one domain, and exhibit a high degree of concordance in a putative nuclear localization sequence (FIG. 1). The RNA products are expressed to an increased extent in the rat brain (hippocampus) following a maximal electric shock, for example. The functional data point to the gene products being involved in neuronal cell death. Apart from being activated by an electric shock, expression of the genes can also be activated by other stimuli which, for example, induce stress, for example by kainate and/or pentylenetetrazole injections.

The conserved, cysteine-rich region in the proteins according to the invention resembles the metallothioneins (=MTs), which exhibit cysteine clusters, possess a small molecular weight and are able to bind metals as a chelate complex (FIG. 2) [Moffatt et al., (1997) Drug Metab. Rev. 29: 261-307]. In the mouse, 4 metallothionein genes have thus far been identified in a 50 kb region on chromosome 8 whereas, in humans, at least 16 MT genes are present as a cluster on chromosome 18. Mouse MT I and MT II are expressed ubiquitously throughout development and are regulated by metals, glucocorticoids and inflammatory stress signals. MT III is mainly expressed neuronally. MT IV is expressed in epithelial cells. In unicellular organisms, the MTs mainly bind copper whereas they bind zinc in mammals, with it being possible for the zinc to be replaced with copper and cadmium ions. Cellular cadmium resistance is achieved by multiplying the MT locus. MTs are probably used functionally as chaperones for metalloproteins and as a reservoir for metals and/or prevent metal toxicity. MT III knockout mice exhibit an increased susceptibility to kainate-induced epileptic seizures and increased neurotoxicity toward kainate in particular regions of the hippocampus, whereas the overexpression of MT III protects mice from kainate-induced cell death [Erickson et al., (1997), J. Neurosci. 17: 1271-1281]. Overexpression of MT I has a protective effect against certain animal models for ischemia (van Lookeren Campagne et al., (1999), Proc. Natl. Acad. Sci. U.S.A. 96: 12870-12875). Metallothioneins are in the main expressed in the cytosol and display their protective effect in this location.

The homology between the L100 gene family, and its homologs, and the metallothioneins is restricted to the short cysteine-rich regions such that L100 and its homologs form an independent family and cannot be regarded as belonging to the metallothioneins. Homology comparisons with ESTs from the Embl database show that L100 is expressed in Caenorhabditis elegans, Drosophila melanogaster and zebrafish, Danio rerio, and presumably arose at an early stage during evolution. The metallothioneins have a length of about 60 amino acids, with about 39% of the amino acids corresponding to the L100 consensus sequence. The L100 homologs are proteins of from about 535 to 589 amino acids such that L100 homologs form an independent family. In humans and in the rat it is possible to demonstrate the presence of at least 2 L100 homologs (annex 1: known ESTs for L100 homologs in the EMBL database, status on 14.3.00), which do not, however, exhibit any homologies with known proteins apart from in the cysteine-rich domain.

A positive influence can be exerted on pathological processes in animals or in humans by way of influencing L100 expression or influencing the biological activity of the L100 protein or the interaction of the L100 protein with its reaction partners which are present in the cell, for example by way of polyclonal and/or monoclonal antibodies or low-molecular-weight substances. In vitro data show that overexpression of L100 leads to cell death. As described above, IEGs are genes whose expression is very rapidly increased by a stimulus. In neurons, they are functionally involved in learning processes, memory, synaptic transmission and neuronal plasticity.

Advantageous nucleic acids according to the invention encode, for example, the human or murine form of L100 or their homologs, or encode homologs in the rat.

Using the two-hybrid system, the L100 (rat) aminoterminus, which contains a putative coiled coil domain, has been observed to interact with protein phosphatase 1 (rat), septin or zinc finger proteins and other unknown ESTs.

Transfections with L100 expression constructs showed that the L100 protein was located in the cell nucleus shortly after the transfection, with the protein subsequently being located in the cytoplasm. Transfection with L100 has induced apoptosis in the cells which have thus far been investigated. In addition, L100 has been detected in hippocampal neurons following overexpression in dendrites.

The nucleic acid sequence according to the invention, the expression cassette (=nucleic acid construct), the vector or the corresponding protein, can be used to identify proteins which exhibit specific binding affinities for the protein according to the invention. Nucleic acids which encode proteins which exhibit specific binding affinities for the protein can also be identified in this way. Advantageously, the two-hybrid system or other biochemical methods are used, either alone or in combination, for this purpose. In this way, it is possible to identify interaction domains, and thus pharmacotherapeutic intervention points, in the protein according to the invention.

The invention therefore relates to the use of the two-hybrid system, or biochemical methods, for identifying the interaction domains of L100 and to the use of this system or these methods for pharmacotherapeutic intervention.

Protein complexes composed of the L100 protein and at least one other protein which interacts with L100, such as protein phosphatase 1, septin, kelch proteins or zinc finger proteins, constitute another particularly advantageous part of the subject-matter of the invention.

Substances which exhibit a specific binding affinity can be found selectively by carrying out structural analyses on the protein according to the invention.

The L100 sequences which have been described make it possible, with the aid of the two-hybrid system or other assays, to locate the amino acids which are responsible for the interaction and to find substances which can be used to influence the interaction between the two proteins.

Another part of the subject-matter of the invention is therefore a method for finding substances which bind specifically to a protein having the above-described amino acid sequence according to the invention or to a protein complex according to the invention, which method comprises one or more of the following steps:
a) preparing a protein having the amino acid sequence according to the invention, or a protein complex containing the protein having the amino acid sequence according to the invention, by expressing the nucleic acid according to the invention, the expression cassette or the vector in a eukaryotic or prokaryotic organism,
b) incubating the protein according to the invention with the substance to be tested or the substances to be tested,
c) detecting the binding of the substance to be tested to the protein having the amino acid sequence according to the invention.

The binding is detected by measuring the antagonization or agonization of the L100 activity or by measuring the physiological effect of L100.

The invention also relates to these substances which are found by using the above method.

Other embodiments of the invention are a method for finding substances which inhibit or augment the interaction of ligands with the protein or protein complex (=protein heteromer) according to the invention or the proteins having the amino acid sequence according to the invention, or a method for finding substances which inhibit or augment the interaction of the proteins according to the invention with proteins such as protein phosphatase 1 or other signal transduction molecules. The two-hybrid system can be used to detect the interaction of proteins with the amino acids according to the invention.

Furthermore, the methods can be carried out by expressing the proteins in eukaryotic cells and linking to a reporter assay, for example using the gfp protein, or other fluorescence assays for activation of the L100 protein.

The invention furthermore relates to a method for qualitatively and quantitatively determining proteins having the amino acid sequence according to the invention in a biological sample, or sample of another nature, which method comprises one or more of the following steps:
a) incubating a biological sample, or sample of another nature, with an antibody according to the invention, which antibody is specifically directed against L100 proteins or homologs,
b) detecting the antibody/antigen complex,
c) comparing the quantities of the antibody/antigen complex with a quantity standard.

In this connection, the L100-ligand binding is used for the detection (see Example 11).

The protein activity, or the quantity of L100 proteins or homologs, can be determined using antibodies. The invention therefore also relates to a method for quantifying the protein activity or quantity of a protein.

The regulatory sequences of the nucleic acids according to the invention, in particular the promoter, the enhancers, what are termed the locus control regions, and the silencers, or any constituent sequences thereof, can be used for ensuring the tissue-specific expression of this gene and/or other genes. This gives rise to the possibility of expressing the genes in nucleic acid constructs in a neuron-specific manner.

In order to isolate a DNA fragment which contains the regions which regulate the transcription of the nucleic acid sequences according to the invention, the region upstream of the transcription start is initially linked to a reporter gene such as galactosidase or GFP (=green fluorescent protein=gfp) and tested in cells, or in transgenic animals such as mice, to determine whether it leads to the specific expression pattern as claimed in claim 1 (Ausubel et al., 1998). Since cis-regulatory sequences can also, inter alia, be located at a very large distance from the transcription start site, it is advantageous if very large genomic regions are included in the analysis. For the cloning, it can be advantageous to use vector systems having a very high cloning capacity, such as BACs or YACs (bacterial artificial chromosome, yeast artificial chromosome). In this connection, the reporter gene can be inserted into the vector by way of homologous recombination and its expression can be investigated (see, for example, Hiemisch et al., (1997), EMBO J. 16: 3995-4006). Important regulatory elements can be identified by making suitable deletions in the construct and then investigating the effects on the expression of the reporter gene (see, for example, Montoliu et al., (1996), EMBO J. 15: 6026-6034). The regulatory sequences of the nucleic acids according to the invention, in particular the promoter, the enhancers, locus control regions and silencers, or any constituent sequences thereof, can be used for the tissue-specific expression of sequences as claimed in claim 1 and other nucleic acid sequences. This thereby provides the possibility of expressing the genes present in nucleic acids in advantageous constructs in a neuron-specific manner. The construct containing the regulatory sequences can be linked to other cDNAs in order to construct animal models in which the given cDNA is expressed in a region-specific manner (see, for example, Oberdick et al., (1990), Science, 248: 223-226). In this connection, it can also be a matter, for example, of expressing sequence-specific DNA recombinases such as CRE recombinase or FLP recombinase, or their derivatives.

It is possible to use the amino acid sequences according to the invention to generate synthetic peptides which can be used as antigens for producing antibodies. It is also possible to use the entire amino acid sequence, or fragments thereof, for generating antibodies. Antibodies are to be understood as meaning, for example, polyclonal, monoclonal, human or humanized or recombinant antibodies, or fragments thereof, single-chain antibodies and also synthetic antibodies. Antibodies according to the invention, or their fragments, are to be understood, in principle, as meaning all immunoglobulin classes, such as IgM, IgG, IgD, IgE and IgA, or their subclasses, such as the IgG subclasses, or their mixtures. Preference is given to IgG and its subclasses such as IgG₁, IgG₂, IgG₂ₐ, IgG_{2b}, IgG₃ or IgG_{M}. Particular preference is given to the IgG subtypes IgG₁ and IgG_{2b}. Fragments which may be mentioned are all the truncated or modified antibody fragments possessing one or two binding sites which are complementary to the antigen, such as antibody moieties having a binding site which corresponds to the antibody and which is formed from a light chain and a heavy chain, such as Fv, Fab or F(ab′)₂ fragments, or single-stranded fragments. Truncated double-stranded fragments, such as Fv, Fab or F(ab′)₂, are preferred. These fragments can be obtained, for example, enzymically by eliminating the Fc moiety of the antibodies using enzymes such as papain or pepsin, by means of chemical oxidation or by means of genetically manipulating the antibody genes. It is also advantageously possible to use genetically manipulated fragments which have not been truncated. The antibodies or fragments can be used either on their own or in mixtures. The antibody genes for the genetic manipulations can be isolated from the hybridoma cells, for example, in a manner known to the skilled person (Ausubel et al., 1998). For this, antibody-producing cells are grown and, when the optical density of the cells is adequate, the mRNA is isolated from the cells, in a known manner, by, for example, disrupting the cells with guanidinium thio-cyanate, acidifying with sodium acetate, extracting with phenol, chloroform/isoamyl alcohol, carrying out precipitations with isopropanol, and washing with ethanol. Reverse transcriptase is then used to synthesize cDNA from the mRNA. The synthesized cDNA can be inserted either directly, or after genetic manipulation, for example by means of site-directed mutagenesis, or the introduction of inserts, inversions, deletions or base substitutions, into suitable animal, fungal, bacterial or viral vectors and expressed in the corresponding host organisms. Preference is given to bacterial, fungal or yeast vectors, such as pBR322, pUC18/19, pACYC184, lambda or yeast mu vectors, for cloning the genes and expressing them in bacteria, such as E. coli, or in yeast, such as Saccharomyces cerevisiae. Specific antibodies directed against the proteins according to the invention are suitable both for use as diagnostic reagents and as therapeutic agents in association with syndromes which are characterized, inter alia, by changes in neural cells.

In addition, the cDNA, the genomic DNA, the regulatory elements of the nucleic acid sequences according to the invention, and also the polypeptide, and constituent fragments thereof, can be used, in recombinant or non-recombinant form, for working out a test system. This test system is suitable for measuring the activity of the promoter or of the protein in the presence of the test substance. In this connection, it is preferably a matter of simple measuring methods (colorimetric methods, luminometric methods, methods based on fluorescence or radioactive methods) which enable a large number of test substances to be measured rapidly (Böhm et al., (1996), “Wirkstoffdesign [Active compound design]”, Spektrum-Verlag, Heidelberg). The described test systems enable chemical or biological libraries to be screened for substances which have agonistic or antagonistic effects on proteins having the amino acid sequence according to the invention or on the protein complex which contains at least one protein according to the invention and at least one further protein which interacts with this protein.

An alternative route for developing active compounds which act on L100 consists in rational drug design (Böhm et al., (1996), “Wirkstoffdesign [Active compound design]”, Spektrum-Verlag, Heidelberg). In this connection, the structure, or a constituent structure, of the protein having the amino acid sequence according to the invention, insofar as this structure is available, or a structural model generated by computers, is used in order, with the support of molecular modeling programs, to find structures which can be predicted to have a high affinity for L100. These substances are then synthesized and tested. High-affinity, selective substances are tested for their use as drugs for treating epilepsies, ischemia, Alzheimer's disease and related dementias, Parkinson's disease, amyotrophic lateral sclerosis, Huntington's disease, multiple sclerosis and other neurodegenerative diseases.

Determining the quantity, activity and distribution of the protein according to the invention, or of its underlying mRNA, in the human body can be used for diagnosing, comprehending predisposition and monitoring in connection with particular diseases. Furthermore, the nucleic acid sequence according to the invention, and its genomic DNA, can be used for drawing conclusions with regard to the genetic causes of, and predispositions for, particular diseases. Both DNA/RNA probes and antibodies of the widest possible variety can be used for this purpose. In this connection, the nucleotide sequence which has been described, or parts thereof, is/are used in the form of suitable probes for uncovering point mutations or deletions/insertions/rearrangements.

The present nucleic acid sequence according to the invention, the expression cassette or the vector, and also the functional equivalents, homologs or derivatives of the nucleic acids, the protein which is encoded by it and which has the amino acid sequence according to the invention, or the protein heteromer (=protein complex) according to the invention containing one of the proteins presented in Example 15, and also reagents (oligonucleotides, antibodies and peptides) derived therefrom, can be used for diagnosing and treating neurodegenerative diseases.

Furthermore, it is possible to monitor the treatment of diseases. This relates, for example, to assessing the course of diseases, to assessing the success of therapies and to graduating a disease.

The invention furthermore relates to a method for qualitatively and quantitatively detecting a nucleic acid according to the invention in a biological sample, which method comprises the following steps:
a) incubating a biological sample with a known quantity of nucleic acid according to the invention or a known quantity of oligonucleotides which are suitable for use as primers for amplifying the nucleic acid according to the invention, or mixtures thereof,
b) detecting the nucleic acid according to the invention by means of specific hybridization or PCR amplification,
c) comparing the quantity of hybridizing nucleic acid, or of nucleic acid which has been obtained by PCR amplification, with a standard or quantity standard.

In addition, the invention relates to a method for qualitatively and quantitatively detecting a protein heteromer according to the invention, or a protein according to the invention, in a biological sample, which method comprises the following steps:
a) incubating a biological sample with an antibody which is specifically directed against the protein heteromer or against the protein according to the invention,
b) detecting the antibody/antigen complex,
c) comparing the quantities of the antibody/antigen complex with a quantity standard.

As the standard, a biological sample is customarily withdrawn from a healthy organism.

The invention furthermore relates to a method for finding substances which bind specifically to a protein having the amino acid sequence according to the invention or to a protein complex which [lacuna] at least one protein according to the invention and at least one other protein which interacts with the protein according to the invention, which method comprises one or more of the following steps:
a) preparing a protein having the amino acid sequence according to the invention, or a protein complex according to the invention, by expressing a nucleic acid according to the invention, an expression cassette or a vector in a eukaryotic or prokaryotic organism,
b) incubating the protein according to the invention, or the protein complex, with the substance to be tested or the substances to be tested,
c) detecting the binding of the substance to be tested to the protein having the amino acid sequence according to the invention, or to the protein complex, or detecting an effect on receptor function.

In addition, the invention relates to a method for finding substances which bind specifically to the protein according to the invention, or to the protein complex, and thereby elicit inhibitory or activating functional effects on L100 signal transmission in neurons.

In situations in which the activity of the protein according to the invention, or of the protein complex, is deficient, several methods can be used for replacing them. In the first place, the protein can be administered naturally or recombinantly, directly or by means of suitable measures, in the form of its encoding nucleic acid (i.e. DNA or RNA). Both viral and nonviral vehicles can be used for this purpose. Another route is provided by using suitable substances to stimulate the endogenous gene. Such substances can be found, for example, by determining their effect on the transcriptional elements of the L100 gene.

In situations in which the activity of the protein according to the invention, or of the protein complex, is in excess, it is possible to make use of specific, synthetic or natural, competitive or noncompetitive, antagonists directed against the protein, or of antibodies or antibody fragments directed against the protein or directed against the protein heteromer. Furthermore, it is possible to inhibit L100 activity, or the activity of the protein, both by using antisense molecules or ribozymes or oligonucleotides and by using low-molecular-weight compounds. Short nucleic acid fragments of from 15 to 100 nucleotides, preferably of from 15 to 40 nucleotides, particularly preferably of from 15 to 25 nucleotides, are advantageously used as antisense molecules.

The nucleic acids according to the invention, or complementary nucleic acid sequences which are derived therefrom, can be used for preparing pharmaceuticals for treating diseases which can be influenced positively by modulating the expression of the L100 gene. Proteins according to the invention, protein fragments or peptides or parts thereof, can be used in just the same way. The invention also relates to the use of antibodies or antibody fragments, or antibody mixtures, which are directed against the protein according to the invention or against the protein heteromer, for producing pharmaceuticals for treating diseases which can be influenced positively by modulating the activity or the quantity of L100 protein. Substances which bind specifically to the protein according to the invention or to the proteins according to the invention or the protein complex can be used for producing pharmaceuticals for treating diseases which can be influenced positively by modulating the activity or quantity of L100 protein. These diseases or disorders are, in the widest possible sense, diseases which are associated with apoptosis and/or neurodegenerative diseases such as epilepsy, ischemia, dementia, Parkinson's disease, Huntington's disease, Alzheimer's disease or CNS trauma. They are preferably diseases such as epilepsy, Alzheimer's disease, ischemia, stroke and CNS trauma.

In addition to this, the nucleic acid sequences according to the invention can also be expressed in the form of therapeutically or diagnostically suitable fragments. Vector systems or oligonucleotides which extend the cDNA by particular nucleotide sequences and consequently encode modified polypeptides which simplify purification can advantageously be used for isolating the recombinant protein. What are termed tags are known, for example, in the literature as being anchors of this nature, for example the hexahistidine anchor, as are epitopes which can be recognized as antigens of a variety of antibodies (described, for example, in Harlow, E. and Lane, D., 1988, Antibodies: A Laboratory Manual. Cold Spring Harbor (N.Y.) Press). These anchors can be used for attaching the proteins to a solid support such as a polymeric matrix, which can, for example, be packed in a chromatography column, or to a microtiter plate or to another support. At the same time, these anchors can also be used for recognizing proteins. For recognizing the proteins, it is also possible to use customary labels such as fluorescent dyes or enzyme labels, which form a detectable reaction product after reacting with a substrate, or a radioactive label, either alone or in combination with the anchors, for derivatizing the proteins. The nucleic acid sequences according to the invention can also be used as markers for human or animal hereditary diseases or for gene therapy.

The DNA-binding property of L100, as a potential transcription factor, can be demonstrated in a manner known to the skilled person, for example by means of bandshift assay [synonyms: gelshift assay, gel retardation assay and electrophoretic mobility shift assay (=EMSA)].

The binding of the protein to the radioactively labeled DNA results in the molecular weight of the complex being increased. This leads to a retardation in the speed of migration of the complex as compared with that of the separate DNA molecule (gel retardation) in a gel electrophoresis run. Following autoradiography, this results in the band being shifted to a higher molecular weight.

The MCKay assay can be used as a further demonstration: an antibody which does not impair the DNA binding of the protein is incubated with cell nucleus extracts and radioactive DNA and then immunoprecipitated. The proteins are removed by phenol extraction and the DNA which remains is analyzed electrophoretically. The radioactivity which is measured is a measure of the affinity of the protein for the DNA. Affinities of transcription factors for their target DNA are from about 10⁸ to 10¹⁴ M⁻¹. If the binding site for L100 on the target DNA is to be characterized, DNA footprinting experiments are then carried out. The principle of DNase I in-vitro footprints is that DNA to which a protein is bound is protected from degradation by DNase I. Molecular analysis of transcription factors showed that these factors comprise a domain which is responsible for the sequence-specific recognition of the DNA and binding to the DNA and another domain which is responsible for the interaction with the basal transcriptional machinery [Papavassiliou A G (1998), Mol. Med. Today 4: 358-366]. Active substances are therefore able to modulate the activity of transcription factors by means of the following mechanisms, inter alia:

They can prevent their degradation or the translocation of the transcription factor into the cell nucleus; specific binding to the transcription factor could prevent interaction with other factors in the cell nucleus; it could be possible to prevent binding to the promoter of regulated target genes [Papavassiliou A G (1997), Mol. Med. 3: 799-810, Papavassiliou A G (1998), Mol. Med. Today 4: 358-366, Papavassiliou A G (2000), J. Cancer Res. Clin. Oncol., 126: 117-118]. Neuro-degenerative processes could therefore be positively modulated by way of regulating transcriptional activity [Dai et al., (1999), Stroke 30: 2391-2398; discussion 2398-2399; Schatz et al., (1999), Exp. Brain Res. 127: 270-278; Skaper et al., (1998) Mol. Cell Neurosci. 12: 179-193; Lokensgard et al., (1998), Mol. Neurobiol. 18: 23-33; Grilli et al., (1996), Science 274: 1383-1385; 37, Lee et al., (1995), Proc. Natl. Acad. Sci. U.S.A., 92: 7207-7211].

Determining the quantity, activity and distribution of the protein according to the invention, or of its underlying mRNA, in the human body can be used for diagnosis, determining predisposition and monitoring in connection with particular diseases. In the same way, the sequence of the cDNA and also of the genomic sequence can be used for drawing conclusions with regard to the genetic causes of, and predispositions to, particular diseases. DNA/RNA probes, and unnatural DNA/RNA probes, and also antibodies of the widest possible variety, can be used for this purpose. In this connection, the nucleotide sequence which has been described, or parts thereof, is used, in the form of suitable probes, for uncovering point mutations or deletions/insertions.

The human form of L100 [=SEQ ID NO: 1 (cDNA), SEQ ID NO: 2 amino acid sequence of L100], the genomic sequence of L100 in the mouse, and the L100 sequence in the mouse and the corresponding amino acid sequence [SEQ ID NO: 5, SEQ ID NO: 3 and SEQ ID NO: 4, respectively] and the L100 homologs in humans and the rat [SEQ ID NO: 6=human homolog, SEQ ID NO: 7 human homolog amino acid sequence, SEQ ID NO: 8=rat homolog, SEQ ID NO: 9 rat homolog amino acid sequence] are described in the following examples.

### EXAMPLES

Unless otherwise indicated, the instructions given in Ausubel et al., 1998 and Sambrook et al., 1989 were followed when carrying out the experiments.

### Example 1

### Screening Method for Identifying the Human L100 Sequence SEQ ID NO: 1

SEQ ID NO: 1 was ascertained by repeatedly screening a human hippocampus library (oligodT- and random-primed) in Lambda ZAP (Stratagene). A rat L100 cDNA (2.97 kb in length) (SEQ ID NO: 10) was used as the probe after having been radioactively labeled (random-primed labeling kit from Roche Diagnostics). The hybridization took place in 5×SSC, 5% Denhardt's solution, 0.025% sodium pyrophosphate, 0.1 mg of yeast tRNA/ml and 50% formamide solution, overnight at 40° C. Washing took place at 60° C. using 2×SSC, 0.1% SDS solution.

### Example 2

### Screening Method for Identifying the L100 Genomic Sequence in the Mouse

The 2.97 kb rat L100 fragment (SEQ ID NO: 10) was radioactively labeled and used for hybridizing a mouse genomic cosmid library (129/Ola, RZPD, Berlin). Cosmid DNA was isolated from one positive clone. This clone was verified as being L100-positive by means of a variety of restriction digestions and hybridizations with L100 probes (by comparing the band patterns which were obtained with those from the genomic DNA of mice). Various fragments from the cosmid were subcloned into a plasmid vector and sequenced using a transposon insertion method (GPS-1, New England Biolabs, Beverly, Mass.; USA), and the sequences were then assembled using the SeqMan program (Lasergene, Madison, Wis., USA).

The genomic sequence of mouse L100 is depicted in SEQ ID NO: 5. The mouse L100 cDNA, which was generated on the basis of the homology with the rat, is depicted in SEQ ID NO: 3. Comparing the mouse genomic sequence with the rat cDNA sequence indicates that the region encoding the L100 protein in the mouse is interrupted by introns. The precise exon/intron structure is shown in FIG. 16.

### Example 3

### Screening Method for Identifying the Sequence of the L100 Homolog in Humans

SEQ ID NO: 1 was used to identify related ESTs in the rat and in humans with the aid of the BLAST program [BALSTN 2.0.11, Altschul et al., (1997), Nucleic Acid Research, 25: 3389-3402]. Primers were derived from the ESTs AI205148 and AA305194; probes were then amplified from hippocampal cDNA (human) and radioactively labeled; the human hippocampus library (oligodT- and random-primed, Stratagene) employed in Example 1 was then screened under the conditions specified in Example 1. A positive Lambda clone contained the open reading frame for SEQ ID NO: 6.

### Example 4

### Screening Method for Identifying the Sequence of the L100 Homolog in the Rat

The complete sequence SEQ ID NO: 8 was identified by using SEQ ID NO: 6, as the radioactively labeled probe, to repeatedly screen, under the conditions described in Example 1, a rat hippocampus library (oligo(dT)-primed, in UniZAP, Stratagene), (Yamagata et al., 1993, Neuron 11: 371-386) which was prepared from rats which had been subjected to repeated electric shocks in the presence of cycloheximide (20 mg/kg i.p.).

### Example 5

### In-Situ Hybridizations of Rat L100

The open reading frame of rat L100 (SEQ ID NO: 10) and 300 base pairs from the 3′ untranslated region were cloned into the vector pBS and the plasmid was linearized in the region of the multicloning site. L100-specific sense and antisense cRNA probes were prepared from this template using T3 RNA polymerase and T7 RNA polymerase, digoxigenin-labeled UTP and standard NTPs. Frozen rat brains and rat embryos were cut, at −20° C., into 15 μm-thick sections in a Cryostat (Leica GmbH), fixed with 4% paraformaldehyde in PBS, permeabilized in 0.2% Triton-X 100, and then hybridized with antisense and sense L100 cRNA probes, overnight at 65° C., in 50% formamide, 5×SSC. These sections were washed with 0.2×SSC at various temperatures and the dioxigenin label was detected using a specific antibody (Kuner et al., (1999), Science, 283, 74-77). Since the sense probes did not yield any signals, it can be assumed that the antisense signals were specific L100 signals. The rat L100 mRNA is expressed ubiquitously and weakly in the rat embryo and expressed strongly in liver, lung, brain, retina and peripheral neurons. (FIG. 3).

During the postnatal development of the brain, rat L100 mRNA is strongly expressed in the hippocampus, the cerebellum, the bulbus olfactorius, the striatum, the cortex and the amygdala (days 4 and 7). In the adult animal, expression of L100 is downregulated when development of the brain has been concluded.

L100 mRNA is expressed in very small quantities in adult brain neurons and cannot be detected in astrocytes.

### Example 6

### Analysis of the Expression of Human L100

The fragment from SEQ ID NO: 1 was labeled radioactively with α-³²P-dCTP using a random-primed labeling kit (Boehringer Mannheim). A Northern blot (in each case 10 μg of human total RNA: brain, heart, skeletal muscle, large intestine, thymus, spleen, kidney, liver, small intestine, placenta, lung and peripheral blood leukocytes) was hybridized, at 68° C. for one hour, with the denatured radioactive probe in QuickHyb solution (Stratagene) and then washed at 60° C. with 0.1×SSC solution. After a screen had lain on the blot for one day, a 3 kb band was detected in a Fuji Phosphoimager FLA2000, with this band indicating that L100 is weakly expressed constitutively in the adult human heart, skeletal muscle, placenta, lung and leukocytes (no figure).

Analysis of the Expression of L100 Under Stress

### Example 7

### Expression of L100 in Rat Brain Following Convulsions

Injections of kainate constitute a recognized animal model for frontal lobe epilepsy. 1.5 hours after the intraperitoneal injection of kainate (10 mg/kg in PBS i.p.), L100 was massively upregulated in the cerebellum, the hippocampus, the cortex and the thalamus. It was possible to observe maximal upregulation in cerebella granular cells and some neurons of the entorhinal, frontal and orbital cortex and hippocampus for up to 6 hours following the injection of kainate (FIG. 4). Normal values were reached once again 24 hours after the injection (FIG. 4). Induction of L100 was also observed following pentylenetetrazole administration (=PTZ, 50 mg/kg in PBS i.p.), with this induction inhibiting the GABAergic neurotransmission which has an inhibitory effect. This results in the animal undergoing epileptic seizures. 20 minutes after PTZ administration, the expression of L100 was increased in cerebella neurons as well as in the thalamus and midbrain cell nuclei and also in the parietal and temporal cortex.

Zinc Neurotoxicity:

The possibility of zinc having a pathogenic role during selective cell death following transient, global ischemia has been discussed in the specialist literature. Zinc is stored in glutamatergic, synaptic vesicles in the brain, released during synaptic activation and taken up by postsynaptic neurons in the cortex and the hippocampus. Zinc has been shown to accumulate, following kainate injections, in some neuronal populations of the outer layer of the hippocampal gyrus dentatus, of the entorhinal cortex and of the cerebellum and also in some hippocampal pyramidal cells. At the same time, these cell populations express L100 mRNA (FIG. 8). The rapid upregulation of L100 in superexcited neurons which accumulate zinc during the convulsion phases suggests that L100 plays a crucial role in excitatory signal cascades which lead to cell death or which seek to prevent cell death. Tests were therefore carried out to determine whether L100 can bind zinc, since metal-binding motifs, such as the cysteine-rich domain, are present in L100.

### Example 8

### L100 Upregulation in the Kindling Model

What is Termed the Kindling Model is a Recognized Animal Model for Epileptogenesis.

After it had been established that L100 mRNA can be induced in the hippocampus following the systemic administration of acute convulsive doses of kainate, it was then necessary to investigate what happens to L100 mRNA expression in a model of epileptogenesis. For this, what is termed the electrical kindling model was investigated in the rat. In a stereotactic operation, a bipolar electrode was implanted into the right baso-lateral amygdala in male Sprague-Dawley rats (aged 3 months). Beginning 2 weeks after the operation, the rats were stimulated electrically once a day by way of the electrode. This electrical stimulation was a fixed intensity pulse (500 μA, 1 msec-long rectangular pulses of 50 Hz for a period of 1 sec). Daily repetition of the stimulation gave rise to what is termed the kindling effect, i.e. the stimulus of constant strength elicited convulsions which increased in duration and severity as the stimulation continued. With the scheme which was used, these convulsions were progressively developing focal convulsions which then generalize secondarily. The convulsion thresholds were determined in accordance with [Racine, R. J., (1972), Electro-encephalogr. Clin. Neurophysiol., 32, 281-294]. The stimulation was carried out until convulsion stage 2 was reached (SS2; facial clonus and/or involuntary nodding of the head) or until there were 3 repeated stage 5 convulsions (SS5; erect position with loss of the labyrinthine reflexes; generally clonic convulsions). 2 hours after the last stimulation, the animals were sacrificed painlessly by decapitation and the total RNA was isolated from the ipsilateral hippocampus in accordance with [Chomczynski et al., (1987), Anal. Biochem. 162, 156-159]. Animals into which electrodes had been implanted but which were not stimulated (sham animals) served as controls. The kindling model was performed in cooperation with Prof. W. Löscher's group (Hanover Veterinary University).

The RNA from 4 animals was pooled in the case of each experimental group. 3 RNA pools were used for the subsequent investigation. Five micrograms of hippocampus total RNA were transcribed into first-strand cDNA in a reverse transcription using a T₁₈(G/A/C)N primer and using Superscript II (Life Technologies) in accordance with the manufacturer's protocols. Quantitative PCR reactions using a LightCycler (Roche) were carried out for measuring the relative quantity of L100 mRNA in the cDNAs. The PCR method used is a method which is carried out in capillaries and in which the amplification of the DNA can be measured by detecting the intercalated SYBR Green fluorescent dye online, and quantitative conclusions can be drawn with regard to the number of template molecules. The amplified products were detected using SYBR Green (Molecular Probes). The PCR reactions were carried out in conformity with the instructions in the protocols provided by the manufacturer of the DNA master SYBR Green (Roche). For amplifying L100, 60 cycles were carried out using an annealing temperature of 65° C. and a final MgCl₂ concentration of 5 mM and employing the following primers:

| | |
|---|---|
| Primer 1: | 5′-GACCATCGGTGATCAAAACTC-3′ |
| Primer 2: | 5′-ACATCAGCTAATGAAGGGCATA-3′ |

For standardizing, a further PCR was carried out using primers for the ribosomal protein S26, the expression of whose gene remains unaltered. For amplifying S26, 60 cycles were carried out using an annealing temperature of 65° C. and a final concentration of MgCl₂ of 5 mM and employing the following primers:

| | |
|---|---|
| Primer 1: | 5′-AAGTTTGTCATTCGGAACATTGT-3′ |
| Primer 2: | 5′-CACCTCTTTACATGGGCTTTG-3′ |

In the quantitative PCR, two dilutions of each cDNA pool were compared with a serial dilution of a standard and the relative quantity of L100 cDNA and/or S26 cDNA in each pool was determined in this way. Care was taken to ensure that all the measured values lay within the concentration range of the standards.

The results are shown in FIG. 5. FIG. 5 depicts the mean values of all three pools per group together with their standard deviations (as a ratio of L100 concentration to S26 concentration). It can be clearly seen that the quantity of L100 mRNA in the ipsilateral hippocampus correlates with the advancing duration and severity of the convulsions. In this connection, the increase at 2 hours after the stage 5 convulsions is statistically significant when compared with the unstimulated control (n=3; p<0.05).

### Example 9

### Upregulation of L100 Following the Administration of Glutamate

If L100 is a marker for overstimulated neurons, which is something which can be deduced from the kainate injections and the increased level of L100 expression in the kindling model, it will also be expected that L100 would be upregulated following glutamate receptor activation.

For this reason, quantitative RT-PCR studies were carried out using cortical neurons which were differentiated following 14 days of in-vitro culture. Glutamate, at a concentration of 100 μM in HSS, was added to neurons for 5 minutes, resulting in excitatory cell death being induced after 18-24 hours. Control cultures were treated with HSS on its own. After that, all the cultures were washed and the conditioned medium was changed. 6 hours after administering glutamate, the RNA was isolated from the neurons using small Quiagen RNeasy columns. 1 μg of total RNA was employed for the cDNA synthesis and this RNA was transcribed using Gibco BRL reverse transcriptase (Superscript II) and permutated hexamers as primer. This single-stranded cDNA was used in a LightCycler™, Roche Molecular Biochemicals, as the template for the PCR. The mixture without the reverse transcriptase was used as the negative control. The values which were measured for L100 were standardized to the quantity of cyclophilin, a gene whose expression is not altered by administering glutamate. The MgCl₂ concentration which was optimal for the L100 PCR was determined and the cDNA was used diluted 1:1, 1:2, 1:4 and 1:8 in H₂O. The L100 sense primers GAA GAG GAA GTT TGA CCA GCT GGA and the L100-antisense primers AGT CCT CCT CTA CAG AAG CGT CAT are located in the 5′ region of L100. They are located on different exons in the genomic DNA and separated by a 2.6 kb intron. It is possible in this way, by means of the choice of the primers, to prevent genomic DNA impurities in the RNA preparation from generating falsely positive signals. Based on cyclophilin, L100 was upregulated by the factor 1.8026±0.555 (N=5). These results show that, following stress, and probably following glutamate receptor activation, L100 is upregulated in neurons prior to glutamate-induced cell death. It is already known that the immediate early genes Fos and Jun are important downstream mediators of long-term effects following glutamate activation. For this reason, L100 may also possibly be able to play an important role in pathophysiological cascades of glutamatergic activation.

### Example 10

### Analysis of the Expression of the L100 Homolog in the Rat

The conditions which were selected in Example 6 were also used for the following experiments. SEQ ID NO: 8 was hybridized, as a probe, with a Northern blot (in each case 10 μg of total RNA from the hippocampus of rats repeatedly subjected to electric shocks or control animals without any electric shock treatment) at 680° C. for 1 hour, and the blot was washed at 60° C. with 0.1×SSC solution. After the screen had lain on the blot for 24 hours, it was possible, using a Fuji Phosphoimager FLA2000, to demonstrate that the quantity of mRNA had increased by a factor of 4 in the hippocampus, after the animals had been killed 4 hours after the last electric shock (FIG. 6). On a blot containing 10 μg of total RNA from rat brain, from rat liver, from rat heart, from rat kidney and from rat testes, SEQ ID NO: 8 was shown to be expressed in a brain-specific manner.

In-Situ Hybridizations of SEQ ID NO: 8 in Rat Tissues

The mRNA for SEQ ID NO: 8 was detected in the adult rat brain using the above-described in-situ hybridization and is distributed uniformly in the brain, with stronger signals for the SEQ ID NO: 8 mRNA being present in the cerebellum, the cortex and the hippocampus. In the hippocampus and gyrus dentatus, SEQ ID NO: 8 is expressed in the CA3 and CA4 regions, in particular, but not in the CA1 section. A moderate upregulation of SEQ ID NO: 8 can be seen in the cerebellum and the cortex following treatment with 4× MECS (FIG. 7). A strong signal was now observed in the CA1 sector of the hippocampus. There was no change in the expression of the gene in rats following treatment with kainate or PTZ.

In summary, it can be stated that, while L100 and L100 homologs do not appear to be absolutely essential for the proper functioning of the adult rat brain in the normal, physiological state, they play an important role during the development of the brain and have essential importance in pathological states which are accompanied by a massive, neuronal overexcitation of the brain.

### Example 11

### Metal-Binding Property of L100

The production and isolation of N-terminal GST-L100 fusion protein were achieved by cloning the SEQ ID NO: 10 open reading frame, without the first methionine of L100, into the vector pGEX-6P. The recombinant protein was induced in protease-free bacteria (SG200.43+pDMI.1) by adding 200 μM IPTG for 3 hours, after which the bacterial pellet was isolated by centrifuging at 3000×g for 20 min and then sonicated in PBS and 0.5% EDTA in the presence of a proteinase inhibitor cocktail supplied by Roche Diagnostics, and subsequently pelleted once again at 6000×g for 10 min. The supernatant was centrifuged once again at 10,000×g and the L100-GST and GST protein extracts, respectively, were affinity-purified through the glutathione-sepharose column supplied by Pharmacia Biotech. The column was eluted, under metal-free conditions, with 10 mM glutathione in 50 mM tris-HCl. The protein content was determined using the BCA assay and the degree of purity was determined using Coomassie-stained polyacrylamide gels. The zinc content in recombinant L100-GST or GST was determined using N-(6-methoxy-8-quinolyl)-p-toluenesulfonamide (TSQ, Molecular Probes). TSQ only fluoresces when zinc is present in complex-bound form and can therefore be used for quantitatively detecting zinc. Equal concentrations of L100-GST or GST were dotted onto glass slides and treated with a 0.05% solution of TSQ (Molecular Probes) in acetone, air-dried and analyzed under a fluorescence microscope at an excitation of 330 nm and an emission of 385 nm. TSQ on its own did not give any fluorescence signal while GST protein gave a weak signal and GST-L100 gave a significantly stronger signal. The weak GST signals could be contaminations with metal-binding proteins from E. coli, with the strong GST-L100 signal demonstrating that L100 is formed in E. coli as a zinc-complexing protein (FIG. 9).

### Example 12

### Subcellular Location of L100 in Mammalian Cells

In order to enable L100 to be detected immunocyto-chemically, the first methionine of the open rat L100 reading frame was replaced with specific, strongly antigenic tags, namely:

| | |
|---|---|
| Myc- | (Met-Glu-Gln-Lys-Leu-Ile-Ser-Glu-Glu-Asp- |
| | Leu) |
| | or |
| Flag- | (Met-Asp-Tyr-Lys-Asp-Asp-Asp-Asp-lys) tag. |

Studies of the heterologous expression of L100 constructs containing an aminoterminal Flag or Myc tag in mammalian cells were used for subcellular location studies. HEK293 cells were incubated, at 37° C., 5% CO₂ and 95% atmospheric humidity, in standard MEM medium (Gibco BRL), 10% fetal calf serum (Sigma Immuno-chemicals) and transfected with L100 plasmids using the calcium phosphate method. At various time intervals after transfecting HEK cells with L100 Flag or empty vector, as the control, the cells were washed with PBS, fixed for 10 min with 2% paraformaldehyde (Sigma Chemicals) and 0.2% glutaraldehyde (Sigma Chemicals), permeabilized for 5 min with 0.2% triton-X-100 (Sigma Chemicals) and then blocked for 30 min with 4% natural goat serum (NGS, Jackson Immunoresearch Labs Inc.). After having been incubated for 2-3 hours with primary anti-Flag M2 antibody (Eastman Kodak Company) in 2% NGS at room temperature, the cells were washed three times, for in each case 10 min, with 1% NGS in PBS and then incubated for 30 min with fluorescence-labeled second antibody (FITC-conjugated anti-mouse antibody, 7.5 μg/ml in 2% NGS); after that, they were washed twice, for in each case 10 min, with PBS and once with 10 mM tris-HCl, pH 7.6, and the coverslips were subsequently sealed with aqueous mounting medium (Sigma Chemicals). The preparations were examined under a fluorescence microscope (Zeiss Axiophot) using a standard filter set. Hoechst 33342 (Molecular Probes) was used as the dye for staining the cell nuclei. Immunocytochemical experiments in HEK293 cells showed that the labeled protein was expressed in the cell nucleus at from 6 to 12 hours after transfection. This observation is in agreement with the nuclear localization signal being present in the L100 sequence (FIG. 10). The carboxy-terminal moiety of L100 carries a high negative charge, something which is regarded as being a characteristic feature of transcriptional activators. The nuclear localization sequence and the negatively charged residues in the C terminus are evolutionarily conserved and make it probable that L100 functions as a transcription factor.

At later times, the L100 protein accumulated in the cytoplasm of the transfected HEK293 cells and could be detected obviously in the cellular protuberances from the cell soma, which protuberances resembled apoptotic bodies in appearance. At the same time, it was possible to observe a condensation of the chromatin scaffolding in the L100-transfected cells, which then died. It was of no importance for the proapoptotic phenotype whether L100 was provided with a tag or not. As the negative control, cells were transfected either with the empty vector or control proteins (GBR2-flag, Kuner et al., (1999), Science, 283, 74-77) (FIG. 11).

In order to obtain direct information about the subcellular location and function of L100 in the brain, primary neurons were transfected with the labeled L100 sequence. These neurons were isolated from rat hippocampi on embryonic days 18 and 19, dissociated and cultured, at 37° C., 5% CO₂ and 95% relative atmospheric humidity, on plates coated with poly-L-lysine (Sigma Chemicals, 0.1%) and laminin (Sigma Chemicals 1-2 82 g/cm2) in a medium containing 1% horse serum, 3% glucose, standard additives and hormones. After one day in vitro, the neurons develop processes and, from 8 to 9 days later, they develop spines which contain synapses. For the expression studies, the neurons were cultured in vitro for 4 days and, after that, transfected with Myc-L100-DNA or control DNA which was complexed with the lipophilic agent Effectine (Qiagen GmbH). After that, the cells were fixed, permeabilized and stained using a monoclonal antibody directed against the Myc epitope (Invitrogen, 4 μg/ml) and an anti-mouse FITC-conjugated second antibody (Jackson Immunoresearch Labs, 7.5 μg/ml), as was described above. At 15 hours after transfection, the myc-L100 was for the most part located in the cell nucleus, and partially located in the cytoplasm, of the neurons (FIG. 12). After 18 hours, L100 was mainly detected in the cytoplasm and the axons. Colocalization with the synaptic marker synaptotagmin (FIG. 13) (specifically stained using rabbit polyclonal anti-synaptotagmin antibody, Chemicon GmbH, and TRITC-conjugated anti-rabbit antiserum, 7.5 μg/ml) was demonstrated in this manner. Expression of L100 in the axons and the dendritic spines could mean that L100 has an important role in post-synaptic signal cascades. Since L100 contains a nuclear localization sequence, L100 could function as a messenger from the synapse to the cell nucleus or vice versa.

### Example 13

### Characterizing L100-Produced Cell Death

For the purpose of further characterizing L100-induced cell death, HEK293 cells were transfected with L100 or control plasmid and counterstained with FLUOS-conjugated annexin (Boehringer Mannheim), a dye which stains phosphatidylserines which form part of the cell wall. In contrast to healthy cells, apoptotic cells expose the phosphatidylserines and are stained with FLUOS-annexin. In order to be able to exclude false positives which are simply due to the cell wall being destroyed in necrotic cells, the apoptotic cells were counterstained with propidium iodide (Boehringer Mannheim). L100 cells were in the main propidium iodide-negative and exhibited stronger staining with FLUOS-annexin (FIG. 14). The negative controls substantiate once again that apoptosis is induced by the heterologous overexpression of L100 in cells.

Effects of L100 Overproduction in Transfected, Primary Hippocampal Neurons:

Cell nucleus stainings were carried out using a Hoechst dye (33342, Molecular Probes) and, at 18 hours after transfection with L100, showed a condensation of the DNA, which is a sign of cell degeneration (FIG. 15). In comparison with the control vector, L100 induced cell death, which was detected by what is termed TUNEL staining. The method detects the free 3′-OH groups after the DNA has become fragmented. The neurons were transfected either with the L100-Myc tag expression plasmid or with the Clontech EYFP expression vector, as described above. At various times after transfection, the cells were washed with PBS and preincubated with TUNEL dilution buffer (Roche Diagnostics, in 30 mM tris-HCl, 140 mM sodium cocodylate and 1 mM cobalt chloride) and then subjected to further incubation with terminal deoxynucleotidyl transferase (Roche Diagnostics) together with biotinylated dNTPs in the TUNEL dilution buffer, at 37° C. for 60 min, in accordance with the manufacturer's instructions.

The cells were washed three times for 5 min with PBS, blocked for 15 min with 10% NGS, incubated for 25 min with TRITC-streptavidin (Jackson Immunoresearch Labs), counterstained with Hoechst 33342, washed for 2×5 min with PBS and sealed in aqueous mounting medium. Analysis under a fluorescence microscope showed that the cell nuclei were stained blue, and the neurons which were identified as having been transfected with L100-Myc tag or EYFP were examined with regard to TUNEL staining (red fluorescence).

In the case of the L100-transfected neurons, it was possible to demonstrate that the number of TUNEL-positive cells increased progressively (FIG. 17). The number of TUNEL-positive cell nuclei was higher in the case of the L100-transfected neurons than in the case of the EYFP-transfected neurons. This shows that, while overexpression of L100 induces cell death 24 hours after the neurons have been transfected, overexpression of a control protein does not do this.

### Example 14

### Producing L100-Transgenic Animals

Important additional information about the (patho)-physiological mechanisms in which the L100 gene is involved can be obtained by selectively mutating the L100 gene in the mouse germ line and analyzing the resulting phenotype. In order to produce what is termed a knock-out mouse, i.e. a mouse which lacks a functional L100 protein, what is termed a targeting construct was first of all prepared. For this, two genomic fragments flanking the L100 coding region (corresponding to positions 2866 to 4083 and 9813 to 13500 in the sequence SEQ ID NO: 5 according to the invention) were cloned, as homology arms for the homologous recombination in embryonic stem cells (ESs), into the vector pHM2 (Kaestner et al., (1994), Gene, 148, 67-70). This vector carries a neomycin resistance cassette and enables a reporter gene to be inserted into the allele to be mutated. For this, the lacZ reporter gene of the vector was fused to the 5′-untranslated region of L100 and was consequently under the control of the endogenous L100 promoter. The lacZ cassette now completely replaces the L100 open reading frame (FIG. 16). After the targeting construct has been linearized, the DNA is to be electroporated into embryonic stem cells and G418-resistant clones are to be selected. Genomic DNA is to be isolated from these clones and to be examined, by means of what is termed nested PCR, for homologous recombination between the targeting construct and the endogenous L100 allele. FIG. 16 shows the genomic structure of L100 and the homology arms which were selected for the homologous recombination, and also the primers for the PCR detection of the recombination. Control constructs containing primer 1 (2728-2749) and as (4064-4083), and also primer 2 (2767-2786) and as (4064-4083), were cloned into pHM2 such that the PCR can be tested with primer 1 or 2 and antisense primers from the lacZ region. Such a PCR can be used to establish, when employing genomic DNA as the template, whether recombination has taken place in the ES cells. As a positive control, genomic DNA can be spiked with these plasmids and the sensitivity of the PCR method can then be determined in this way.

### Example 15

### Protein-Protein Interactions of L100

Amino Acid Structure of L100:

L100 encodes a protein of 581 amino acids. The L100 protein possesses a cysteine-rich domain (235-273) which exhibits 33% identity with metallothioneins, a potential coil-coil domain (119-155), and a serine-rich region (18-42) and a glutamate-rich region (402-407). In addition, it is also possible to demonstrate the presence of a potential nuclear localization signal (200-207) which could be responsible for the transport of L100 into the nucleus (SEQ ID NO: 10) (FIG. 1).

Two-Hybrid Search Using the Carboxy Terminus and Amino Terminus of L100:

The cDNA encoding the carboxy terminus of the L100 gene (SEQ ID NO: 11, amino acids 407-581) was amplified from L100 rat cDNA in a PCR (polymerase chain reaction) using the specific L100 primers L100-5′-Y2H-407 (ACAGCGACGTCGACG-GAGGGAGTGTGGGCAACTT) and L100-3′Y2H-581 (ATAGTTTAGCGGCCGCTT-ACACAGGCACAGGGTC), restricted with the enzymes NotI and SalI and, after that, cloned into the vector pDBleu (from Gibco), which had previously been cut with NotI/SalI. The resulting construct (L100-407-581) encodes a protein in which the Gal4 binding domain is fused to the C terminus of the L100 gene.

The cDNA encoding the amino terminus of the L100 gene (SEQ ID NO: 11, amino acids 2-315) was amplified from L100 rat cDNA in a PCR using the specific L100 primers L100 5′-2-Y2H (CAAACGCGTCGACCGCTGGGATAC-AGAAGAAG) and L100-3′-315-Y2H (ATAGTTTAGCGGCCGCTTAGTCCTCCATGGG-GGACTC) and also cloned into the vector pDBleu (from Gibco) after having been subjected to restriction digestion with the enzymes NotI and SalI. In this construct (L100-2-315), the Gal 4 binding domain was fused to the N terminus of L100. The constructs L100-407-581 and L100-2-315 were transformed into the yeast strain Y190.

The yeast strains resulting from this were tested for self-activation and the concentration of 3-amino-triazole (3AT) which is required for basal HIS3 expression was determined. HIS3 encodes imidazole glycerol phosphate dehydratase, which is an enzyme of histidine biosynthesis. Weak protein-protein interactions can be identified by determining the threshold value for 3-AT resistance.

The yeast strains were transformed with a rat hippocampus cDNA library prepared from MECS-induced rats, which library is cloned into the vector pPC86 (from Gibco). 4×10⁶ transformants were plated out on tryptophan/leucine/histidine deficient medium containing an appropriate 3-AT concentration (20 mM 3-AT). After 3, 4 and 5 days of growth at 30° C., colonies were isolated and stained with XGAL. A total of 19 colonies were found to be His3 and lacZ positive in the case of the L100 N terminus while 6 colonies were found to be His3 and lacZ positive in the case of the L100 COOH terminus. The pPC86 plasmid DNA was purified from the positive yeast colonies and transformed, for amplification, into the E. coli strain Xl1blue. The resulting pPC86 DNA from the positive colonies was cotransformed with various pDBleu constructs into the yeast strain Y190.

In the case of the N terminus, activation of the reporter genes His3 and lacZ in combination with the construct L100-2-315 was only observed in 3 out of the 19 colonies. In the case of the C terminus, 1 colony exhibited activation of the reporter genes His3 and lacZ in combination with the construct L100-407-581. The positive cDNAs from the vector pPC86 were amplified using the vector-specific primers pPC86a (GTATAACGCGTTTGGAATCAC) and pPC86b (GTAAATTTCTGGCAAGGTAGAC), and the resulting PCR fragment was sequenced.

In the case of the L100 N terminus, it was possible to identify 3 interaction partners:

1) Protein phosphatase 1 alpha (13 x,=PP1). PP1 is expressed ubiquitously and is involved in a large number of metabolic processes such as muscle contractions, cell cycle and neurotransmission (Ohkura et al., (1989), Cell 57: 997-1007; Kitamura et al., (1991), J. Biochem., 109: 307-310; Sasaki et al., Jpn. J. Cancer Res., (1990), 81: 1272-1280). There are 2 main isoforms, i.e. PP1 alpha and delta, which possess virtually identical catalytic domains and only differ in their NH₂-termini and COOH-termini (Andreassen et al., (1998), J. Cell Biol., 14: 1207-1215). Furthermore, PP1 is concentrated in the dendrites and plays a role, inter alia, in the activity of the AMPA channels, in the dopamine regulation of the Ca2+flow and the neuropeptide regulation of the K+flow [Smith F. D. et al., (1999), J. Biol. Chem. 274, 28, 19894-19900; Yan et al., (1999), Nature neuroscience, Vol. 2, No 1; Allen P. B. et al., (1997), Proc. Natl. Acad. Sei. USA 94, 9956-9961, Terry-Lorenzo et al., (2000), J. Biol. Chem., 275: 2439-2446; Strack et al., (1999), J. Comp. Neurol., 413: 373-384; Osterhout et al., (1999), J. Cell Biol. 145: 1209-1218; Schillace et al., (1999), Curr. Biol. 9: 321-324; Kasahara et al., (1999), J. Biol. Chem., 274: 9061-9067; Evans et al., (1999), Eur. J. Neurosci., 11: 279-292; Collins et al., (1998), Methods Mol. Biol., 93: 79-102; Yan et al., (1997), Neuron 19: 1115-1126; Strack et al., (1997), Brain Res. Mol. Brain Res. 49: 15-28; 16. Fernandez-Sanchez et al., (1996), FEBS Lett. 398: 106-112; Younossi-Hartenstein et al., (1996), J. Comp. Neurol. 370: 313-329; Papasozomenos et al., (1995), J. Neurochem. 65: 396-406; Saito et al., (1995), Biochemistry 34: 7376-7384; Veeranna et al., (1995), J. Neurochem. 64: 2681-2690; Vickroy et al., (1995), Neurosci. Lett. 191: 200-204; da Cruz e Silva et al., (1995), J. Neurosci. 15: 3375-3389; Ouimet et al., (1995), Proc. Natl. Acad. Sci. U.S.A. 92: 3396-3400; Hashikawa et al., (1995), Neurosci. Res. 22: 133-136; Surmeier et al., (1995), Neuron 14: 385-397; Kotter R. (1994), Prog. Neurobiol. 44: 163-196; Ulloa et al., (1993), FEBS Lett. 330: 85-89]. SEQ ID NO: 12 gives the sequence of protein phosphatase 1 alpha.

2) Identifying an L100 binding partner possessing a kelch motif

The sequence of another binding partner is thus far unknown; it contains a 55% amino acid sequence identity with “kelch” proteins (Xue et al., 1993 Cell, 72(5): 681-693) and a 33% identity with the mouse zinc finger protein 151 (Jordan-Sciutto et al., (1999), J. Biol. Chem. 274: 35262-35268; Schulz et al., 1995, BIOCHEM. J. 311: 219-224). The keich repeats, consisting of 50 amino acids, possess the ability to bind actin (Field et al., 1999, Trends Cell. Biol., 9 (10): 387-394; Kim et al., 1999, Gene, 228(12): 73-83; Hernandez et al., 1997, J. Neurosci., 17(9): 3038-3051). Kelch proteins can possess a POZ domain in the N terminus, which domain is involved, inter alia, in chromatin folding and the organization of the cytoskeleton in brain cells (Ahmad et al., 1998, Proc. Natl. Acad. Sci. U.S.A., 95(21): 12123-12128). The identified sequence possesses 67.4% nucleotide sequence identity in 350 nt with Mayven, an actin-binding protein which is mainly expressed in primary neurons of the hippocampus. Mayven contains a BTB/POZ domain and kelch repeats and is colocated with actin filaments in stress fibers and cortical actin-rich regions of the cell margins (Soltysik-Espanola et al., 1999, Mol. Biol. Cell., 10(7): 2361-2375). The sequence of the identified L100 interaction partner containing the kelch repeat is given in sequence SEQ ID NO: 13.

Sequence comparison with kelch Drosophila melanogaster ring canal protein (kelch protein)

Length=689, Score=130 bits (323), Expect=3e-30, Identities=63/114 (55%), Positives=83/114 (72%)

| | |
|---|---|
| NPAHMGKAFKVMNELRSKRLLCDVMIVAEDVEVEAHRVVLAACSPYFCAMFTGDMSESKA | 436 |
| N H ++F MNE+R ++ LCDV++VA+DVE+ AHR+VLA+CSPYF AMFT ES+ | |
| NEQHTARSFDAMNEMRKQKQLCDVILVADDVEIHAHRMVLASCSPYFYAMFT-SFEESRQ | 194 |
| KKIEIKDVDGQTLSKLIDYIYTAEIEVTEENVQVLLPAASLLQLMDVRRTAVTF | 598 |
| +I ++ VD + L LIDY+YTA +EV E+NVQVLL AA+LLQL DVR | |
| FARITLQSVDARALELLIDYVYTATVEVNEDNVQVLLTAANLLQLTDVRDACCDF | 248 |

Sequence comparison with mouse zinc finger protein 151

Length=749, Score=72.6 bits (175), Expect=6e-13, Identities=36/106 (33%), Positives=63/106 (58%)

| | |
|---|---|
| PAHMGKAFKVMNELRSKRLLCDVMIVAEDVEVEAHRVVLAACSPYFCAMFTGDMSESKAK | 439 |
| P H + + +N+ R LLCD V + V+ +AH+ VLAACS YF +F + + | |
| PQHSQRVLEQLNQQRQLGLLCDCTFVVDGVDFKAHKAVLAACSEYFKMLF---VDQKDVV | 60 |
| KIEIKDVDGQTLSKLIDYIYTAEIEVTEENVQVLLPAASLLQLMDV | 577 |
| ++ I + G L ++++++YTA++ ++ ENV +L AS LQ+ D+ | |
| HLDISNAAG--LGQVLEFMYTAKLSLSPENVDDVLAVASFLQMQDI | 104 |

For review: [Field et al., (1999), Trends Cell. Biol. 9: 387-394]

3) The sequence of the 3rd interaction partner for the L100 N terminus is unknown; it was not possible to detect any homology with protein binding motifs. The sequence possesses homology with a human sequence on chromosome 17 (homo sapiens chromosome 17, clone hRPK.2).
Identities=134/146 (91%)

One interaction partner was identified in the case of the L100 COOH-terminus. This gene exhibits 95% sequence identity (605 bp) with Septin 6 (Kinoshita M., “Identification of mouse Septin6 gene and its product”; while the sequence is in the EMBL database, the paper relating to it has still not been published). It is thought that Septin 6 belongs to the Septin family. This family consists of GTPases which are able to form plasma membrane-associated filaments. Septin complexes may play an important role in vesicle transport and in the organization of the proteins at the neuron plasma membrane [Hsu et al., (1998), Neuron 20: 1111-1122]. Septins are involved, inter alia, in cyto-kinesis and the organization of the cytoskeleton and are found in neurofibrilla tangles in Alzheimer patients [Kinoshita et al., (1998), Am. J. Pathol. 153: 1551-1560]. SEQ ID NO: 14 shows the nucleic acid sequence of Septin 6. [Fung et al., (1999), FEBS Lett. 451: 203-208; Kinoshita et al., (1998), Am. J. Pathol. 153: 1551-1560; Caltagarone et al., (1998), Neuroreport 9: 2907-2912; Hsu et al., (1998), Neuron 20: 1111-1122; Fares et al., (1995), Mol. Biol. Cell. 6: 1843-1859].

1. An isolated nucleic acid sequence which encodes a polypeptide possessing apoptosis activity and which is selected from the group consisting of:
a) a nucleic acid sequence having the sequence depicted in SEQ ID NO: 1, SEQ ID NO: 3, SEQ ID NO: 5, SEQ ID NO: 6 or SEQ ID NO: 8,
b) nucleic acid sequences which, as a result of the degeneracy of the genetic code, are derived from the nucleic acid sequence depicted in SEQ ID NO: 1, SEQ ID NO: 3, SEQ ID NO: 5, SEQ ID NO: 6 or SEQ ID NO: 8,
c) derivatives of . the nucleic acid sequence depicted in SEQ ID NO: 1, SEQ ID NO: 3, SEQ ID NO: 6 or SEQ ID NO: 8 which encode polypeptides having the amino acid sequences depicted in SEQ ID NO: 2, SEQ ID NO: 4, SEQ ID NO: 7 or SEQ ID NO: 9 and which exhibit at least 90% homology on the amino acid level without the biological activity of the polypeptides being significantly reduced.

2. An amino acid sequence encoded by a nucleic acid sequence as claimed in claim 1.

3. An amino acid sequence as claimed in claim 2 which is encoded by the sequence depicted in SEQ ID NO: 1, SEQ ID NO: 3, SEQ ID NO: 5, SEQ ID NO: 6 or SEQ ID NO: 8.

4. An expression cassette which contains a nucleic acid sequence as claimed in claim 1, with the nucleic acid sequence being linked, in antisense or sequence being linked, in antisense or sense, to one or more regulatory signals.

5. A vector which contains a nucleic acid sequence as claimed in claim 1 or an expression cassette as claimed in claim 4.

6. A recombinant prokaryotic or eukaryotic host organism which harbors at least one nucleic acid sequence as claimed in claim 1 or at least one expression cassette as claimed in claim 4 or at least one vector as claimed in claim 5.

7. A recombinant prokaryotic or eukaryotic host organism as claimed in claim 6, with the organism being a microorganism or an animal.

8. The use of a protein as claimed in claim 2, or peptide fragments thereof, as an antigen for generating specific polyclonal or monoclonal antibodies or antibody mixtures directed against proteins as claimed in claim 2.

9. A polyclonal or monoclonal antibody or antibody mixture which specifically recognizes a protein as claimed in claim 2.

10. The use of a nucleic acid sequence as claimed in claim 1, or a fragment thereof, for isolating a genomic sequence by means of homology screening.

11. The use of a nucleic acid sequence as claimed in claim 1 as a marker for human hereditary diseases.

12. The use of a nucleic acid sequence as claimed in claim 2, of an expression cassette as claimed in claim 4, or of a fragment of these, for gene therapy.

13. A protein complex as claimed in claim 13, characterized in that the other protein is protein phosphatase 1, septin, a kelch protein or a zinc finger protein.

14. A method for finding substances which bind specifically to a protein having an amino acid sequence as claimed in claim 2 or to a protein complex as claimed in claim 12, which method comprises one or more of the following steps:
a) preparing a protein having an amino acid sequence as claimed in claim 2 or a protein complex as claimed in claim 12 by expressing a nucleic acid as claimed in claim 1, an expression cassette as claimed in claim 4 or a vector as claimed in claim 5 in a eukaryotic or prokaryotic organism,
b) incubating the protein as claimed in claim 2 or the protein complex as claimed in claim 12 with the substance to be tested or the substances to be tested,
c) detecting the binding of the substance to be tested to the protein having the amino acid sequence as claimed in claim 2 or to the protein complex as claimed in claim 12.

15. A substance which is found using a method as claimed in claim 14.

16. A substance which inhibits or augments the interaction of associated proteins with proteins having the amino acid sequences as claimed in claim 2.

17. The use of the amino acid sequence as claimed in claim 2, or of the nucleic acid sequence as claimed in claim 1, for computer-assisted modeling for finding and for selectively designing substances which possess specific binding affinity for a protein as claimed in claim 2.

18. A method for qualitatively or quantitatively detecting a nucleic acid as claimed in claim 1 in a biological sample, which method comprises one or more of the following steps:
a) incubating a biological sample with a known quantity of nucleic acid as claimed in claim 1 or a known quantity of oligonucleotides which are suitable for use as primers for amplifying the nucleic acid as claimed in claim 1, or mixtures thereof,
b) detecting the nucleic acid as claimed in claim 1 by means of specific hybridization or PCR amplification,
c) comparing the quantity of hybridizing nucleic acid as claimed in claim 1, or of nucleic acid as claimed in claim 1 which is obtained by PCR amplification, with a standard.

19. A method for qualitatively or quantitatively detecting a protein as claimed in claim 2, or a protein complex as claimed in claim 12, in a biological sample, which method comprises one or more of the following steps:
a) incubating a biological sample with an antibody as claimed in claim 9 which is specifically directed against proteins as claimed in claim 2 or protein complexes as claimed in claim 12,
b) detecting the antibody/antigen complex,
c) comparing the quantities of the antibody/antigen complex with a quantity standard.

20. A method for quantifying the activity or quantity of a protein as claimed in claim 2 using antibodies as claimed in claim 9.

21. The use of nucleic acids as claimed in claim 1, or of complementary nucleic acid sequences which are derived therefrom, for producing drugs for treating diseases which can be influenced positively by modulating expression of the L100 gene.

22. The use of proteins as claimed in claim 2, or of fragments of these proteins, for producing drugs for treating diseases which can be influenced positively by modulating the activity or quantity of L100 protein.

23. The use of antibodies as claimed in claim 9 for producing drugs for treating diseases which can be influenced positively by modulating the activity or quantity of L100 protein.

24. The use of substances as claimed in claim 15 for producing drugs for treating diseases which can be influenced positively by modulating the activity or quantity of L100 protein.
